(19) United States
(12) Patent Application Publication
(10) Pub. No.: US 2023/0270409 A1
(43) Pub. Date: Aug. 31, 2023

(54) ULTRASONIC IMAGE DISPLAY SYSTEM AND STORAGE MEDIA

(71) Applicant: GE Precision Healthcare LLC, Wauwatosa, WI (US)

(72) Inventor: Shunichiro Tanigawa, Hino (JP)

(21) Appl. No.: 18/170,074

(22) Filed: Feb. 16, 2023

(30) Foreign Application Priority Data
Feb. 17, 2022 (JP) 2022-023328

(51) Int. Cl. A61B 8/00; A61B 8/06

(52) U.S. Cl. CPC A61B 8/463; A61B 8/06

(57) ABSTRACT

An ultrasonic diagnostic device includes an ultrasonic probe, a user interface, a display, and one or more processors. The one or more processors execute operations including: selecting a preset used in examination from among a plurality of presets set for a plurality of examination locations based on a signal input through the user interface, deducing an examination location of a subj ect using a trained model by inputting, to the trained model, an input image created based on an ultrasonic image obtained by scanning the subject via the ultrasonic probe, determining whether to recommend that a user change the selected preset to a preset of the deduced examination location based on the selected preset examination location and the deduced examination location; and displaying a message on the display recommending that the user change the preset when it is determined that a preset change should be recommended.

### FIELD OF THE INVENTION

The present invention relates to an ultrasonic image display system in which presets can be changed and to storage media used by the ultrasonic display system.

### BACKGROUND OF THE INVENTION

When scanning a subject using an ultrasonic diagnostic device, prior to starting a scan of the subject, a user checks presets set in advance such as imaging conditions for each examination location and selects a preset corresponding to an examination location of the subject.

A preset includes a plurality of items corresponding to an examination location and the content of each item. The plurality of items has, for example, a setting item relating to a measurement condition such as transmission frequency or gain, a setting item relating to an image quality condition such as contrast, and a setting item relating to a user interface of a display screen.

A preset is set for each examination location, so performing examination of a subject using a preset for an examination location different from that examination location of the subject may lead to difficulty in acquiring an ultrasonic image of a desired image quality. For example, if a selected preset is a mammary gland preset despite the examination location of the subject being the lower extremities, it may be difficult to acquire an image of a desired image quality for the lower extremities. Therefore, the user must change the preset to a preset for the examination location of the subject being examined. However, while examining a subject, the user must perform a plurality of work processes and may start examination of a subject without remembering to change the preset. If the user recalls forgetting to change the preset in the middle of the examination, they will change the preset, but depending on the level of image quality for the ultrasonic images acquired prior to changing the preset, the user may have to restart the examination of the subject over from the beginning, which is a problem in that it increases a burden on the user.

A conceivable method of handling this problem is to deduce the examination location based on an ultrasonic image of the subject, and automatically changing the preset when a current preset set by the user is a preset for a separate examination location different from the examination location of the subject. However, when deduction accuracy is low and the preset is not changed automatically, there is a risk that, conversely, the image quality of the ultrasonic image will be worse.

Therefore, in cases where a preset for a separate examination location different from the examination location of the subject is set, technology is required to assist the user so that examination of the subject can be performed using a preset for the examination location of the subject.

### Brief Description of the Invention

A first aspect of the present invention is an ultrasonic image display system including an ultrasonic probe, a user interface, a display, and one or a plurality of processors for communicating with the ultrasonic probe, the user interface, and the display, wherein the one or a plurality of processors execute operations including: selecting a preset used in examination from among a plurality of presets set for a plurality of examination locations based on a signal input through the user interface; deducing an examination location of a subject using a trained model by inputting to the trained model an input image created based on an ultrasonic image obtained by scanning the subject via the ultrasonic probe; determining whether to recommend that a user change the selected preset to a preset of the deduced examination location based on the selected preset examination location and the deduced examination location; and displaying a message on the display recommending that the user change the preset when it is determined that a preset change should be recommended to the user.

A second aspect of the present invention is non-transitory storage media that can be read non-temporally by one or more computers on which are stored one or more commands that can be executed by one or more processors that communicate with an ultrasonic probe, a user interface, and a display, wherein the one or more commands execute operations including: selecting a preset used in examination from among a plurality of presets set for a plurality of examination locations based on a signal input through the user interface; deducing an examination location of a subject using a trained model by inputting, to the trained model, an input image created based on an ultrasonic image obtained by scanning the subject via the ultrasonic probe; determining whether to recommend that a user change the selected preset to a preset of the deduced examination location based on the selected preset examination location and the deduced examination location; and displaying a message on the display recommending that the user change the preset when it is determined that a preset change should be recommended to the user.

A third aspect of the present invention is a method for recommending a change to a preset using an ultrasonic image display system including an ultrasonic probe, a user interface, and a display, the method including: selecting a preset used in examination from among a plurality of presets set for a plurality of examination locations based on a signal input through the user interface; deducing an examination location of a subject using a trained model by inputting, to the trained model, an input image created based on an ultrasonic image obtained by scanning the subject via the ultrasonic probe; determining whether to recommend that a user change the selected preset to a preset of the deduced examination location based on the selected preset examination location and the deduced examination location; and displaying a message on the display recommending that the user change the preset when it Is determined that a preset change should be recommended to the user.

With the present invention, when a determination is made as to whether to recommend a preset change to a user and it is determined that a preset change should be recommend, a message is displayed on the display recommending that the user change the preset. Therefore, by checking the message, the user can notice that a currently selected preset does not match a preset for the actual examination location of the subject. When a preset change is recommended, the user can change the preset as needed at a time convenient for the user. Furthermore, by recommending the preset change to the user, a final decision as to whether to change the preset can be deferred to the user, so image quality of ultrasonic images conversely being worse due to the preset being changed automatically can be avoided.

### BRIEF DESCRIPTION OF THE DRAWINGS

FIG. 1 is a diagram illustrating a state of scanning a subject via an ultrasonic diagnostic device 1 according to Embodiment 1 of the present invention.

FIG. 2 is a block diagram of the ultrasonic diagnostic device 1.

FIG. 3 is a schematic view of an original image.

FIG. 4 is an explanatory diagram of training data being generated from the original image.

FIG. 5 is an explanatory diagram of the correct data.

FIG. 6 is a diagram illustrating training data mAi, qAj, and rAk, and a plurality of correct data 61.

FIG. 7 is an explanatory diagram of a method for creating a trained model.

FIG. 8 is a diagram illustrating an example of a flowchart executed in an examination of a subject 52.

FIG. 9 is an explanatory diagram of a method for inputting patient information.

FIG. 10 is a diagram illustrating an example of a settings screen for selecting a preset for an examination location of a subject.

FIG. 11 is an explanatory diagram of a preset.

FIG. 12 is a diagram illustrating a button B0 displayed as highlighted.

FIG. 13 is an explanatory diagram of a deduction phase of the trained model 71.

FIG. 14 is a diagram illustrating an aspect of scanning a new subject 53.

FIG. 15 is a diagram illustrating an example of a flowchart executed in an examination of the new subject 53.

FIG. 16 is an explanatory diagram of a deduction phase of the trained model 71.

FIG. 17 is a diagram illustrating an example of a message 86 displayed on a display monitor 18.

FIG. 18 is a diagram illustrating an example of a preset change screen displayed on a touch panel 28.

FIG. 19 is a diagram illustrating a flow of examination of the new subject 53 in Embodiment 2.

FIG. 20 is an explanatory diagram illustrating a flow of examination of the new subject 53 in Embodiment 3.

FIG. 21 is an explanatory diagram of the deduction phase of the trained model 71.

FIG. 22 is an explanatory diagram of the process for Step ST24.

FIG. 23 is a diagram illustrating an example of deduction results for the probability P when TH2 < P.

FIG. 24 is a diagram illustrating an example of a deduction result for the probability P when TH1 ≦ p ≦ TH2.

FIG. 25 is a diagram illustrating a flow of examination of the new subject 53 in Embodiment 4.

FIG. 26 is a diagram illustrating a flow of examination of the new subject 53 in Embodiment 5.

FIG. 27 is a diagram illustrating a flow of examination of the new subject 53 in Embodiment 6.

FIG. 28 is a diagram illustrating an example of deduction results displayed on the display monitor 18.

FIG. 29 is a diagram illustrating another example of the deduction results displayed on the display monitor 18.

FIG. 30 is an example of the deduction results of an examination location displayed in further detail.

FIG. 31 is a diagram illustrating an example in which a color image 88 is displayed.

FIG. 32 is a diagram illustrating an example of a settings screen for setting an operating mode of the ultrasonic diagnostic device.

### DETAILED DESCRIPTION OF THE INVENTION

An embodiment for carrying out the invention will be described below, but the present invention is not limited to the following embodiment.

### Embodiment 1

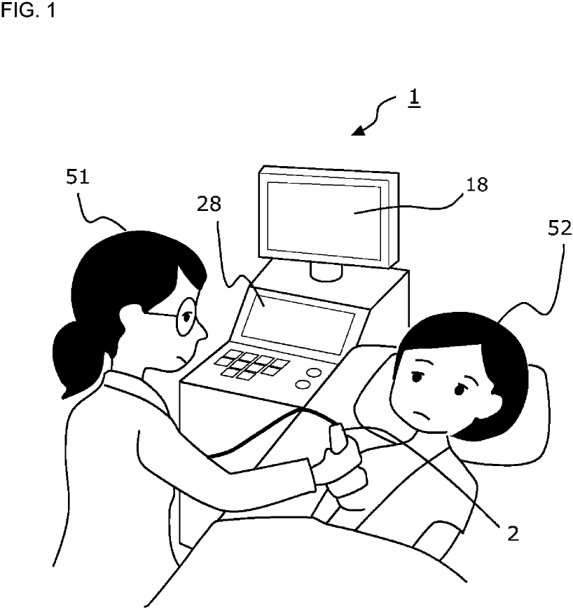

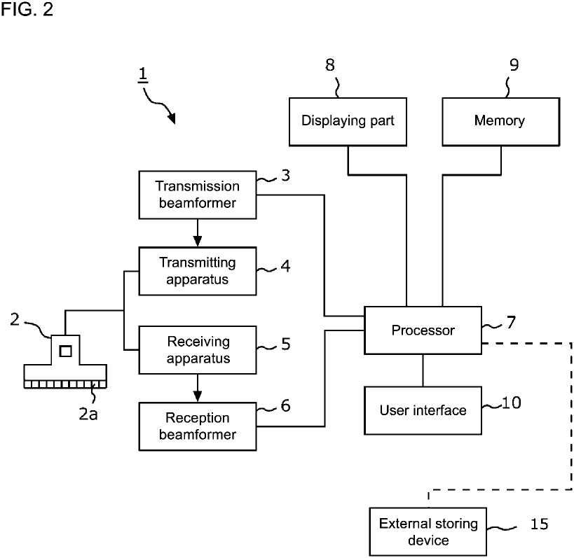

FIG. 1 is a diagram illustrating an aspect of scanning a subject via an ultrasonic diagnostic device 1 according to Embodiment 1 of the present invention, and FIG. 2 is a block diagram of the ultrasonic diagnostic device 1.

The ultrasonic diagnostic device 1 has an ultrasonic probe 2, a transmission beamformer 3, a transmitting apparatus 4, a receiving apparatus 5, a reception beamformer 6, a processor 7, a display 8, a memory 9, and a user interface 10. The ultrasonic diagnostic device 1 is one example of the ultrasonic image display system of the present invention.

The ultrasonic probe 2 has a plurality of vibrating elements 2a arranged in an array. The transmission beamformer 3 and the transmitting apparatus 4 drive the plurality of vibrating elements 2a, which are arrayed within the ultrasonic probe 2, and ultrasonic waves are transmitted from the vibrating elements 2a. The ultrasonic waves transmitted from the vibrating elements 2a are reflected within the subject 52 (see FIG. 1) and a reflection echo is received by the vibrating elements 2a. The vibrating elements 2a convert the received echo to an electrical signal and output this electrical signal as an echo signal to the receiving apparatus 5. The receiving apparatus 5 executes a prescribed process on the echo signal and outputs the echo signal to the reception beamformer 6. The reception beamformer 6 executes reception beamforming on the signal received through the receiving apparatus 5 and outputs echo data.

The reception beamformer 6 may be a hardware beamformer or a software beamformer. If the reception beamformer 6 is a software beamformer, the reception beamformer 6 may include one or a plurality of processors, including one or a plurality of: i) a graphics processing unit (GPU), ii) a microprocessor, iii) a central processing unit (CPU), iv) a digital signal processor (DSP), or v) another type of processor capable of executing logical operations. A processor configuring the reception beamformer 6 may be configured by a processor different from the processor 7 or may be configured by the processor 7.

The ultrasonic probe 2 may include an electrical circuit for performing all or a portion of transmission beamforming and/or reception beamforming. For example, all or a portion of the transmission beamformer 3, the transmitting apparatus 4, the receiving apparatus 5, and the reception beamformer 6 may be provided in the ultrasonic probe 2.

The processor 7 controls the transmission beamformer 3, the transmitting apparatus 4, the receiving apparatus 5, and the reception beamformer 6. Furthermore, the processor 7 is in electronic communication with the ultrasonic probe 2. The processor 7 controls which of the vibrating elements 2a is active and the shape of an ultrasonic beam transmitted from the ultrasonic probe 2. The processor 7 is also in electronic communication with the display 8 and the user interface 10. The processor 7 can process echo data to generate an ultrasonic image. The term “electronic communication” may be defined to include both wired and wireless communications. The processor 7 may include a central processing unit (CPU) according to one embodiment. According to another embodiment, the processor 7 may include another electronic component that may perform a processing function such as a digital signal processor, a field programmable gate array (FPGA), a graphics processing unit (GPU), another type of processor, and the like. According to another embodiment, the processor 7 may include a plurality of electronic components capable of executing a processing function. For example, the processor 7 may include two or more electronic components selected from a list of electronic components including a central processing unit, a digital signal processor, a field programmable gate array, and a graphics processing unit.

The processor 7 may also include a complex demodulator (not illustrated in the drawings) that demodulates RF data. In a separate embodiment, demodulation may be executed in an earlier step in the processing chain.

Moreover, the processor 7 may generate various ultrasonic images (for example, a B-mode image, color Doppler image, M-mode image, color M-mode image, spectral Doppler image, elastography image, TVI image, strain image, and strain rate image) based on data obtained by processing via the reception beamformer 6. In addition, one or a plurality of modules can generate these ultrasonic images.

An image beam and/or an image frame may be saved and timing information may be recorded indicating when the data is retrieved to the memory. The module may include, for example, a scan conversion module that performs a scan conversion operation to convert an image frame from a coordinate beam space to display space coordinates. A video processor module may also be provided for reading an image frame from the memory while a procedure is being implemented on the subject and displaying the image frame in real-time. The video processor module may save the image frame in an image memory, and the ultrasonic images may be read from the image memory and displayed on the display 8.

In the present Specification, the term “image” can broadly indicate both a visual image and data representing a visual image. Furthermore, the term “data” can include raw data, which is ultrasound data before a scan conversion operation, and image data, which is data after the scan conversion operation.

Note that the processing tasks described above handled by the processor 7 may be executed by a plurality of processors.

Furthermore, when the reception beamformer 6 is a software beamformer, a process executed by the beamformer may be executed by a single processor or may be executed by the plurality of processors.

Examples of the display 8 include a LED (Light Emitting Diode) display, an LCD (Liquid Crystal Display), and an organic EL (Electro-Luminescence) display. The display 8 displays an ultrasonic image. In Embodiment 1, the display 8 includes a display monitor 18 and a touch panel 28, as illustrated in FIG. 1. However, the display 8 may be configured of a single display rather than the display monitor 18 and the touch panel 28. Moreover, two or more display devices may be provided in place of the display monitor 18 and the touch panel 28.

The memory 9 is any known data storage medium. In one example, the ultrasonic image display system includes a non-transitory storage medium and a transitory storage medium. In addition, the ultrasonic image display system may also include a plurality of memories. The non-transitory storage medium is, for example, a non-volatile storage medium such as a Hard Disk Drive (HDD) drive, a Read Only Memory (ROM), etc. The non-transitory storage medium may include a portable storage medium such as a CD (Compact Disk) or a DVD (Digital Versatile Disk). A program executed by the processor 7 is stored in the non-transitory storage medium. The transitory storage medium is a volatile storage medium such as a Random Access Memory (RAM).

The memory 9 stores one or a plurality of commands that can be executed by the processor 7. The one or a plurality of commands cause the processor 7 to execute the operations described hereinafter in Embodiments 1 to 9.

Note that the processor 7 may also be configured to be able to connect to an external storing device 15 by a wired connection or a wireless connection. In this case, the command(s) causing execution by the processor 7 can be distributed to both the memory 9 and the external storing device 15 for storage.

The user interface 10 can receive input from a user 51. For example, the user interface 10 receives instruction or information input by the user 51. The user interface 10 is configured to include a keyboard (keyboard), a hard key (hard key), a trackball (trackball), a rotary control (rotary control), a soft key, and the like. The user interface 10 may include a touch screen (for example, a touch screen for the touch panel 28) for displaying the soft key and the like.

The ultrasonic diagnostic device 1 is configured as described above.

When scanning a subject using the ultrasonic diagnostic device 1, the user 51 selects a preset for an examination location of the subject before starting to scan the subject.

A preset is a data set including a plurality of items corresponding to an examination location and the content of each item. The plurality of items has, for example, a setting item relating to a measurement condition such as transmission frequency or gain, a setting item relating to an image quality condition such as contrast, and a setting item relating to a user interface of a display screen.

When examining a subject, the user 51 operates the user interface 10 of the ultrasonic diagnostic device 1 to select a preset for an examination location of the subject. After selecting this preset, the user 51 scans the subject. Once the scan of the subject has ended, the user 51 inputs a signal indicating that the scan of the subject has ended. When this signal is input, the ultrasonic diagnostic device 1 recognizes that the examination of the subject has ended.

Once the examination of the subject has ended, the user 51 performs examination of a next, new subject. When performing the examination of the new subject, the user 51 selects a preset for an examination location of the new subject. If the examination location of the new subject is the same as the examination location of the subject immediately prior, the preset selected during the examination of the subject immediately prior can be used as is. In this case, the user 51 performs the examination of the new subject without changing the preset. Once the examination has ended, the user 51 inputs the signal indicating that the examination of the subject has ended.

Similarly below, each time examination of a subject is performed, the examination of the subject is performed by selecting a preset for an examination location of the subject.

Meanwhile, in clinical settings, ultrasound examinations are extremely important in diagnosing subjects, ultrasound examinations are performed at many medical institutions, and the number of subjects who receive ultrasound examinations during medical checkups or the like is increasing. Therefore, the number of subjects that the user 51 examines on a daily basis is also increasing which, in turn, increases a work load on the user 51. Furthermore, when performing an examination of a subject, the user 51 must perform various work while examining the subject such as probing an examination location while communicating with the subject in order to examine the subject. Thus, when examining a plurality of subjects, the user may forget to change a preset and start examination of a new subject. If an examination location of the new subject is the same as an examination location of a subject immediately prior, examination of the new subject can be subsequently done via the preset used in examination of the subject immediately prior. However, the examination location of the new subject may be different from the examination location of the subject immediately prior. Items included in a preset often differ by examination location and setting values of items differ by examination location as well, so when performing examination of a new subject using a preset for an examination location of a subject immediately prior as is may not allow an ultrasonic image of a desired image quality to be obtained. Therefore, the user 51 must change the preset to a preset for the examination location of the new subject. However, as described above, since the user 51 must carry on performing a plurality of work processes when examining a subject, they may start the examination of the new subject without changing the preset. If the user 51 recalls forgetting to change the preset in the middle of the examination, they will change the preset, but the ultrasonic images prior to changing the preset will have been acquired via the preset for the examination location of the subject immediately prior. Therefore, due to the level of image quality of the ultrasonic image acquired prior to changing the preset, the user 51 will have to start the examination of the subject over from the beginning, which is a problem in that it increases a burden on the user 51.

Changing the preset automatically is conceivable as a method of addressing this problem. However, if the changed preset does not match a preset for an examination location of a subject, there is a risk that conversely the image quality of the ultrasonic image will be worse.

Therefore, the ultrasonic diagnostic device 1 according to Embodiment 1 is configured to recommend a preset change to the user 51 when a selected preset is not a preset for an examination location of an actual subject. One method of recommending a preset change to the user 51 is described below.

Note that in the Embodiment 1, in order to recommend a preset change to the user 51, the ultrasonic diagnostic device 1 primarily executes operations (1) and (2) below.

(1) Recommend an examination location of a subject using a trained model.

(2) Determine whether to recommend a preset change to a user based on the recommendation results in (1).

As described above, in Embodiment 1, an examination location of a subject is recommended using a trained model, and whether to recommend a preset change to a user is determined based on this recommendation result. Therefore, in Embodiment 1, before examining a subject, a trained model suitable for recommending an examination location of a subject is generated. Therefore, first, a training phase for generating this trained model is described below. Following description of this training phase, a method for recommending a preset change to the user 51 is described.

### Training Phase

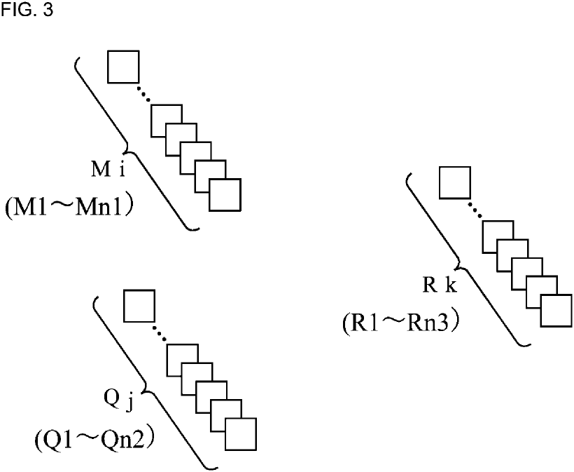

FIGS. 3 to 7 are explanatory diagrams of the training phase.

In the training phase, first, original images are prepared which form a basis for generating training data.

FIG. 3 is a schematic view of the original image.

In Embodiment 1, an ultrasonic image Mi (i = 1 to n1) acquired by a medical institution such as a hospital, an ultrasonic image Qj (j = 1 to n2) acquired by a medical equipment manufacturer, and an ultrasonic image (called “air image” below) Rk (k = 1 to n3) acquired in a state in which the ultrasonic probe is suspended in the air are prepared as the original images.

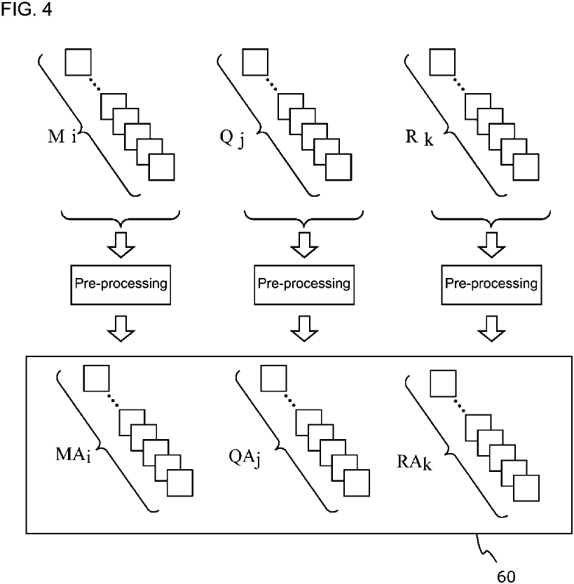

Next, as illustrated in FIG. 4, pre-processing is executed on these original images Mi, Qj, and Rk.

This pre-processing includes, for example, image cropping, standardization, normalization, image inversion, image rotation, a magnification percentage change, and an image quality change. By pre-processing the original images Mi, Qj, and Rk, pre-processed original images MAi, QAj, and RAk can be obtained. Each pre-processed original image is used as training data for creating the trained model. A training data set 60 including the pre-processed original images MAi, QAj, and RAk can be prepared in this manner. The training data set 60 includes, for example, 5,000 to 10,000 rows of training data.

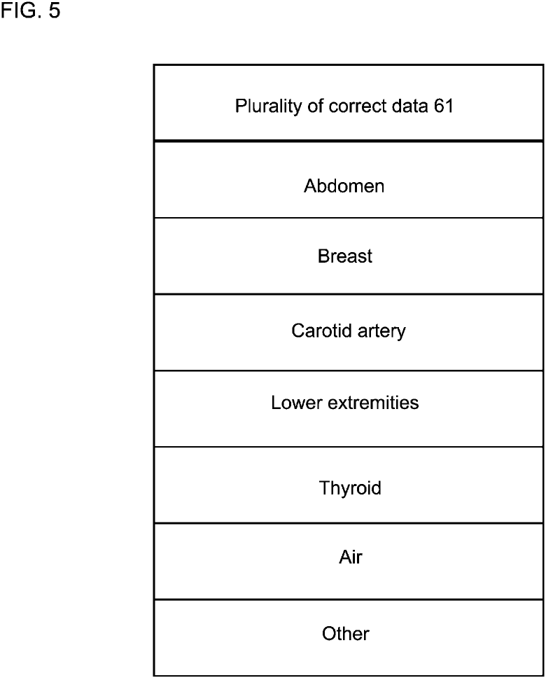

Next, these training data are labelled as correct data (see FIG. 5).

FIG. 5 is an explanatory diagram of the correct data.

In Embodiment 1, a plurality of examination locations targeted for examination via a plurality of the ultrasonic diagnostic devices 1 are used as the correct data.

Although a number of the examination locations targeted for examination or a scope of each examination location is believed to vary by medical institution, here, the following six locations of the human body are considered as the examination locations for the sake of simplifying description.

“Abdomen”, “mammary gland”, “carotid artery”, “lower extremities”, “thyroid”, and “other”.

Note that “other” indicates all locations other than the “abdomen”, “mammary gland”, “carotid artery”, “lower extremities”, and “thyroid”.

Therefore, “abdomen”, “mammary gland”, “carotid artery”, “lower extremities”, “thyroid”, and “other” are included in the plurality of correct data 61 used in Embodiment 1. Furthermore, since the training data generated based on the air image is also included in the training data set 60, correct data indicating the training data to be air is included in the plurality of correct data 61 as well. Therefore, in Embodiment 1, the following seven correct data are considered as the plurality of correct data 61.

“Abdomen”, “mammary gland”, “carotid artery”, “lower extremities”, “thyroid”, “air”, and “other”.

The correct datum “air” indicates that the training data is data generated based on an air image. Furthermore, the correct data “abdomen”, “mammary gland”, “carotid artery”, “lower extremities”, and “thyroid” respectively indicate that an examination location of the training data is the “abdomen”, “mammary gland”, “carotid artery”, “lower extremities”, or “thyroid”. The correct datum “other” indicates that an examination location of the training data is a location other than the “abdomen”, “mammary gland”, “carotid artery”, “lower extremities”, or “thyroid”.

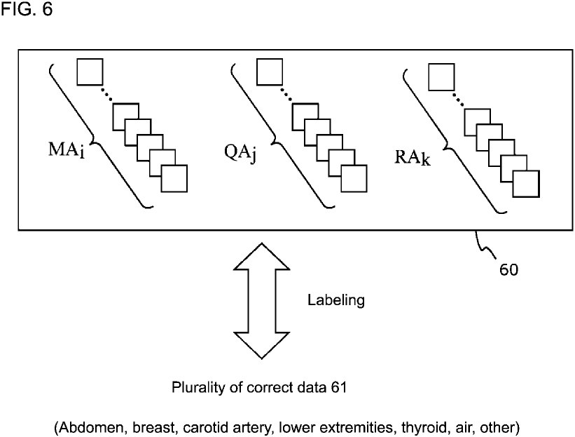

These training data are labelled as correct data. FIG. 6 illustrates the training data MAi, QAj, and RAk, and the plurality of correct data 61. In Embodiment 1, as illustrated in FIG. 6, each training datum is labeled by the corresponding correct datum among the above seven correct data “abdomen”, “mammary gland”, “carotid artery”, “lower extremities”, “thyroid”, “air”, and “other”.

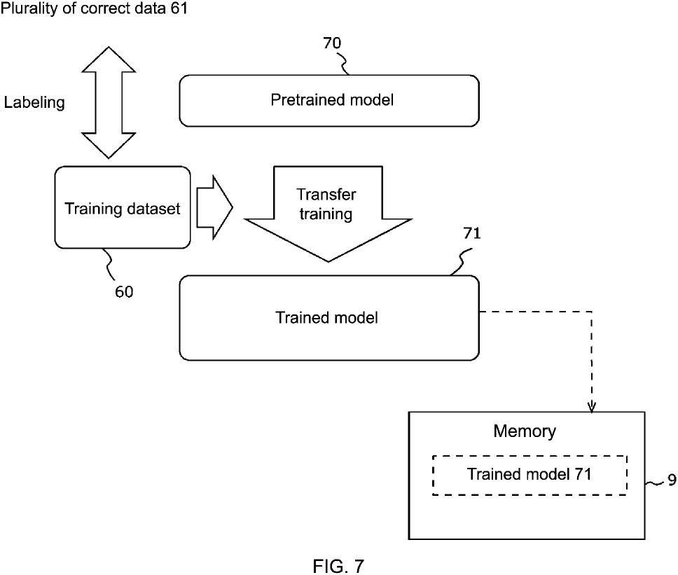

Next, the trained model is created using the above training data (see FIG. 7).

FIG. 7 is an explanatory diagram of a method for creating a trained model.

In Embodiment 1, a trained model 71 is created using transform training technology.

First, a pretrained model 70 is prepared as a neural network. The pretrained model 70 is, for example, generated using an ImageNet data set or created using BERT.

Next, the training data labeled with the correct data is taught to the pretrained model 70 using the transform training technology to create the trained model 71 for recommending an examination location.

After the trained model 71 is created, an evaluation of the trained model 71 is performed. The evaluation may, for example, use a confusion matrix. Accuracy (accuracy), for example, may be used as an indicator index for the evaluation.

If the evaluation is favorable, the above trained model 71 is used as a model for recommending whether a location on a subject or the like is an examination location. If the evaluation is unfavorable, additional training data is prepared and training is performed again.

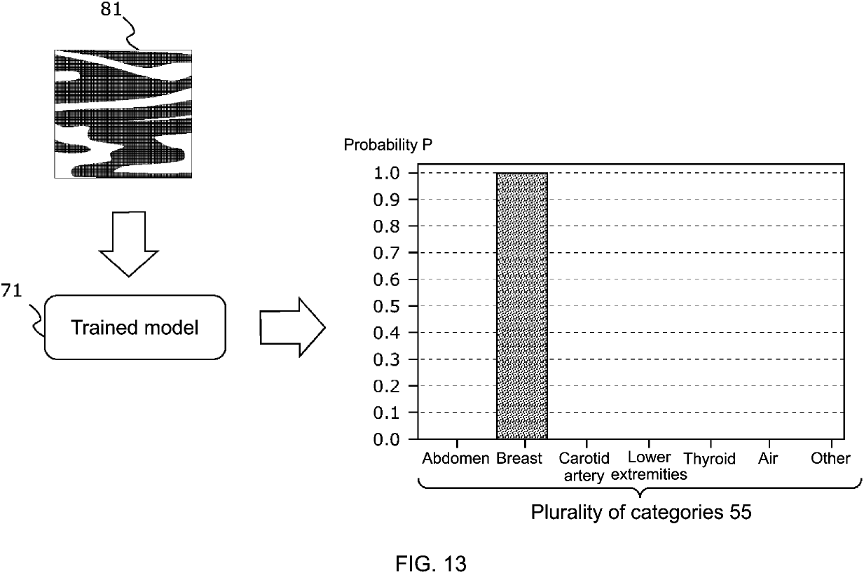

The trained model 71 can be created in this manner. As illustrated in FIG. 13 described hereinafter, the trained model 71 recommends which category an input image 81 is to be classified, selected from a plurality of categories 55 including “abdomen”, “mammary gland”, “carotid artery”, “lower extremities”, “thyroid”, “air”, and “other”. This trained model 71 is stored in the memory 9 of the ultrasonic diagnostic device 1. Note that the trained model 71 may be stored in an external storing device 15 accessible by the ultrasonic diagnostic device 1.

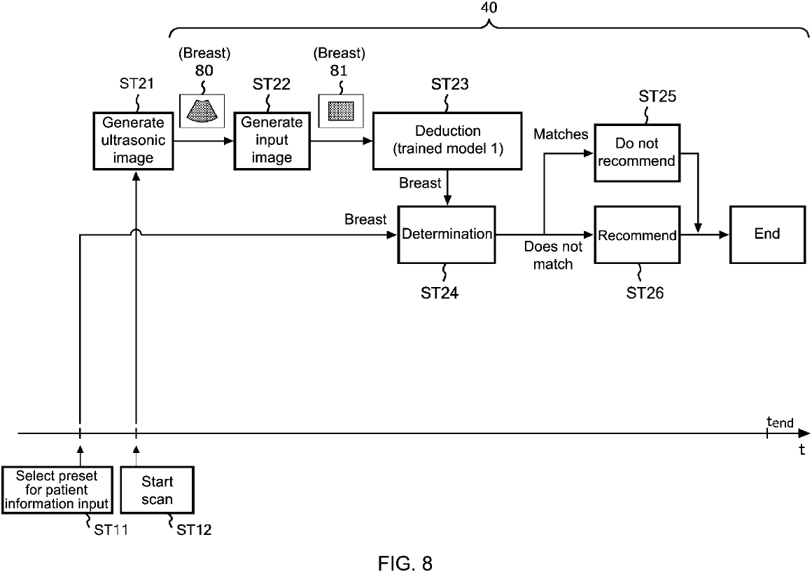

In Embodiment 1, a preset change is recommended to the user 51 using the trained model 71. An example of the recommendation method is described below with reference to FIG. 8.

FIG. 8 is a diagram illustrating an example of a flowchart executed in an examination of a subject 52 (see FIG. 1).

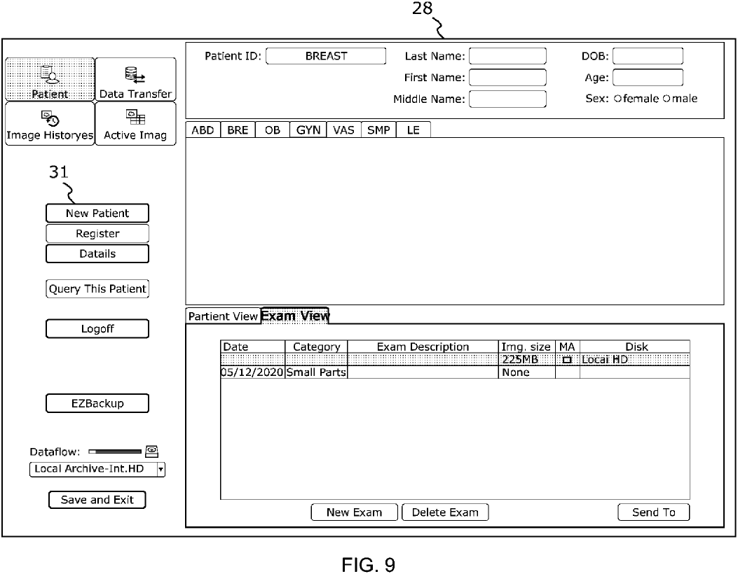

In step ST11, the user 51 leads the subject 52 (see FIG. 1) to an examination room and has the subject 52 lie down on an examination bed. In addition, the user 51 operates the user interface 10 (see FIG. 2) to set each item that must be set in advance before scanning the subject 52. For example, the user 51 operates the user interface 10 to input patient information. FIG. 9 is an explanatory diagram of a method for inputting patient information.

The user 51 displays a settings screen for patient information on the touch panel 28. Once the settings screen is displayed, the user clicks a “new patient” button 31. By this button 31 being clicked, an input screen for patient information is displayed. The user 51 inputs the patient information and other information as needed. For example, when the user 51 clicks the “New patient” button 31 or when input of required patient information is completed, the processor 7 can determine whether a signal indicating the start of the examination of the subject 52 was input. Therefore, for example, the ultrasonic diagnostic device 1 can recognize that the examination of the subject 52 has started by the user 51 clicking the “new patient” button 31. Note that the settings screen for patient information may be displayed on the display monitor 18.

Furthermore, the user 51 operates the user interface 10 to select a preset for the examination location of the subject 52.

A preset includes a plurality of items corresponding to an examination location and the content of each item. The plurality of items includes, for example, a setting item relating a measurement condition such as transmission frequency or gain.

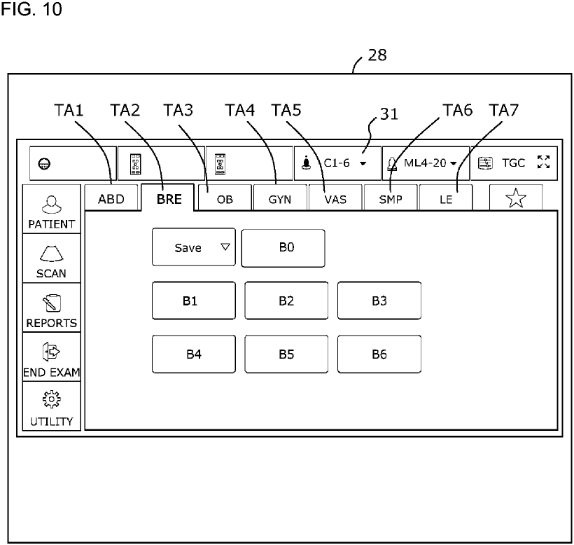

FIG. 10 is a diagram illustrating an example of a settings screen for selecting a preset for an examination location of a subject 52.

The user 51 operates the touch panel 28 to display a settings screen for an examination location. When the user 51 touches the tab 31, a plurality of tabs TA1 to TA7 are displayed on the settings screen. These tabs TA1 to TA7 are classified by examination type. Note that the settings screen for examination location may be displayed on the display monitor 18.

Examples of types of examination by the ultrasonic diagnostic device include abdominal, mammary, cardiovascular, gynecological, musculoskeletal, neonatal, neurological, obstetric, ophthalmological, small parts, superficial tissue, vascular, venous, and pediatric.

In FIG. 10, the tabs TA1 to TA7 are displayed which correspond to a portion of the examination types. The tabs TA1 to TA7 correspond to the abdominal, mammary, obstetric, gynecological, vascular, small parts, and pediatric examination types respectively.

In FIG. 10, an example is displayed in which the mammary tab TA2 is selected.

A plurality of buttons B0 to B6 are displayed in a region of the mammary tab TA2.

Among these buttons B0 to B6, the button B0 displays a button that sets a mammary preset. The remaining boxes B1 to B6 respectively indicate buttons that set a preset for a superior medial mammary gland portion, inferior medial mammary gland portion, superior lateral mammary gland portion, armpit mammary gland portion, inferior lateral mammary gland portion, and areola mammary gland portion.

Clicking the buttons B0 to B6 allows the user 51 to confirm an item set for each examination location and to confirm setting content of the items. For example, by clicking the button B0, the user 51 can confirm a preset including an item set for the examination location “mammary gland” and setting content of the item.

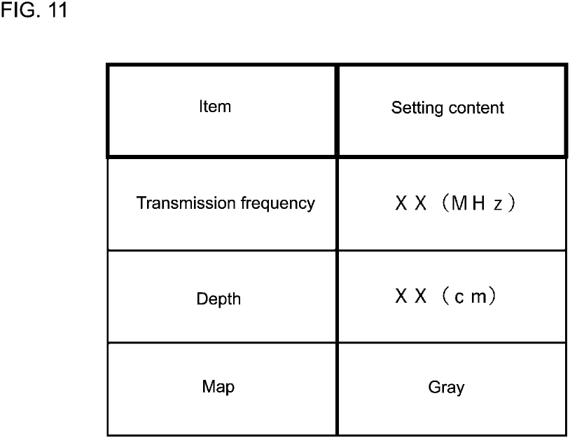

FIG. 11 is an explanatory diagram of a preset.

A preset includes an item corresponding to an examination location and setting content of the item. An item has, for example, a setting item relating to a measurement condition such as transmission frequency or gain, a setting item relating to an image quality condition such as contrast, a setting item relating to a user interface of a display screen, a setting item relating to body marking and probe marking, a setting item relating to image adjustment parameters, and a setting item relating to image conditions.

In FIG. 11, a transmission frequency, depth, and map are illustrated as examples of items corresponding to an examination location.

The setting content for transmission frequency is represented by a specific frequency value (for example, a number of mHz). Also, the setting content for depth is represented by a specific depth value (for example, a number of cm). The setting content for the map is “grey”. Here, the map is represented by a grey display.

Therefore, the user 51 can confirm preset information for the examination location “mammary gland”. Furthermore, the user 51 can change the setting content as needed. For example, the depth can be changed to a different value.

Similarly, upon clicking each of the buttons B1 to B6, the user 51 can confirm a preset, which includes an item corresponding to each of a mammary gland location (superior medial mammary gland portion, inferior medial mammary gland portion, superior lateral mammary gland portion, armpit mammary gland portion, inferior lateral mammary gland portion, and areola mammary gland portion) and setting content thereof. For example, upon clicking the button B6, the user 51 can confirm the item corresponding to the areola portion and the content set for each item.

In the case of examining, as the examination location, a “mammary gland” of the subject 52, the user selects the “mammary gland” preset. On the other hand, when examining only a particular location of the mammary gland rather than the entire mammary gland of the subject 52, a preset for the particular location in question is selected.

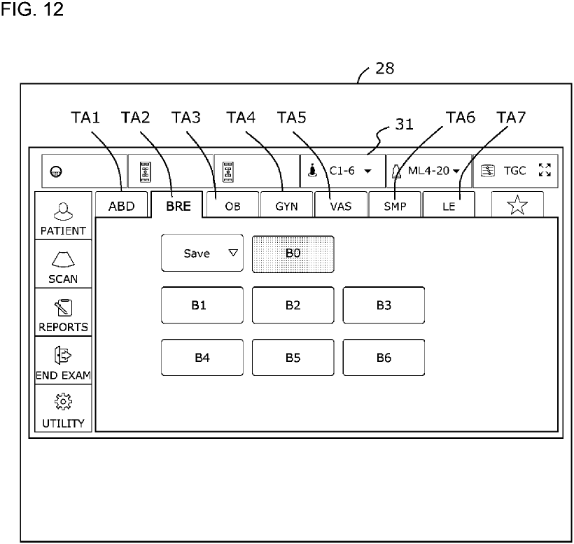

Here, the examination location of the subject 52 is set to “mammary gland”. Therefore, the user 51 selects the mammary gland preset. The user 51 operates the touch panel 28 to input a selection signal for selecting the mammary gland preset. In response to this selection signal, the processor 7 selects the preset for mammary gland. As illustrated in FIG. 12, when this preset is selected, the button B0, which corresponds to mammary gland, is displayed as highlighted. As such, the user 51 can visually confirm that the mammary gland preset is selected.

Therefore, when the user operates the user interface 10 to input a signal for selecting a preset, the processor can select a preset used in examination from the plurality of presets based on this input signal.

Note that in the case that only a particular location of a mammary gland is being targeted for examination rather than the entire mammary gland being targeted for examination, a preset for the particular location may be selected. For example, when the preset for the armpit portion is selected, the button B5 is displayed as highlighted, and when the preset for the areola portion is selected, the button B6 is displayed as highlighted. Here, as described above, the examination location of the subject 52 is “mammary gland”, so the button B0, which corresponds to mammary gland, is displayed as highlighted.

Returning to FIG. 8, description is continued.

In step ST11, once the user 51 has input patient information, selected a preset, and completed operations necessary for another examination, the processor proceeds to step ST12 and scanning of the subject 52 begins.

While pressing the ultrasonic probe 2 against an examination location of the subject 52, the user 51 operates the probe and scans the subject 52. In Embodiment 1, the examination location is the mammary gland so, as illustrated in FIG. 1, the user 51 presses the ultrasonic probe 2 against the mammary gland of the subject 52. The ultrasonic probe 2 transmits an ultrasonic wave and receives an echo reflected from within the subject 52. The received echo is converted to an electrical signal, and this electrical signal is output as an echo signal to the receiving apparatus 5 (see FIG. 2). The receiving apparatus 5 executes a prescribed process on the echo signal and outputs the echo signal to the reception beamformer 6. The reception beamformer 6 executes reception beamforming on the signal received through the receiving apparatus 5 and outputs echo data.

The process next proceeds to step ST21.

In step ST21, the processor 7 generates an ultrasonic image 80 based on the echo data.

The user 51 confirms the generated ultrasonic image 80, stores the ultrasonic image 80 as needed, and the like, and continues performing work for acquiring ultrasonic images.

Meanwhile, the processor 7 executes a process 40 for determining whether to recommend a preset change to the user 51 based on the ultrasonic image 80 acquired in step ST21. The process 40 is described hereinbelow.

In step ST22, the processor 7 generates the input image 81 input to the trained model 71 based on the ultrasonic image 80.

The processor 7 executes pre-processing of the ultrasonic image 80. This pre-processing is basically the same as the pre-processing executed when generating training data for the trained model 71 (see FIG. 4). The input image 81 input to the trained model 71 (see FIG. 7) can be generated by executing the pre-processing. After the input image 81 is generated, the process proceeds to step ST23.

In step ST23, the processor 7 deduces a location shown by the input image 81 using the trained model 71 (see FIG. 13).

FIG. 13 is an explanatory diagram of the deduction phase of the trained model 71.

The processor 7 inputs the input image 81 to the trained model 71 and, using the trained model 71, deduces which location among the plurality of locations of the subject is the location shown by the input image 81. Specifically, the processor 7 deduces into which category the location of the input image 81 is to be classified, selected from the plurality of categories 55 including the “abdomen”, “mammary gland”, “carotid artery”, “lower extremities”, “thyroid”, “air”, and “other”. Moreover, the processor 7 obtains a probability of the location shown by the input image 81 being classified into each category.

Specifically, the trained model 71 obtains, for the location of the input image 81, a probability of being classified as “abdomen”, a probability of being classified as “mammary gland”, a probability of being classified as “carotid artery”, a probability of being classified as “lower extremities”, a probability of being classified as “thyroid”, a probability of being classified as “air”, and a probability of being classified as “other”, and outputs an obtained probability P.

In FIG. 13, a deduction result is output showing that the probability of the location of the input image 81 being the mammary gland (mammary gland) is close to 100%. Therefore, the processor 7 recommends “mammary gland” as the location shown by the input image 81. After deducing the location shown by the input image 81, the process proceeds to step ST24.

In step ST24, the processor 7 determines whether the examination location deduced in step ST23 matches an examination location of the preset selected by the user 51. When the examination locations match, the processor 7 proceeds to step ST25 and determines not to recommend a preset change to the user 51, and the process 40 ends.

On the other hand, when the examination locations do not match, the processor 7 proceeds to step ST26 and determines to recommend a preset change to the user 51.

Here, the examination location of the preset selected by the user 51 is “mammary gland” and the deduced examination location is also “mammary gland”. Therefore, in step ST24, the processor 7 determines that the examination location deduced in step ST23 matches the examination location of the preset selected by the user 51 in step ST11, and the process proceeds to step ST25. The processor 7 determines not to recommend a preset change to the user 51, and the process 40 ends.

Meanwhile, the user 51 scans the subject 52 while operating the ultrasonic probe 2 to acquire an ultrasonic image necessary for examination. Once scanning of the subject is completed, the user 51 operates the user interface 10 to input a signal indicating that examination of the subject has ended. In FIG. 8, the time at which the examination of the subject ended is shown as “t end”. The examination of the subject 52 ends in this manner.

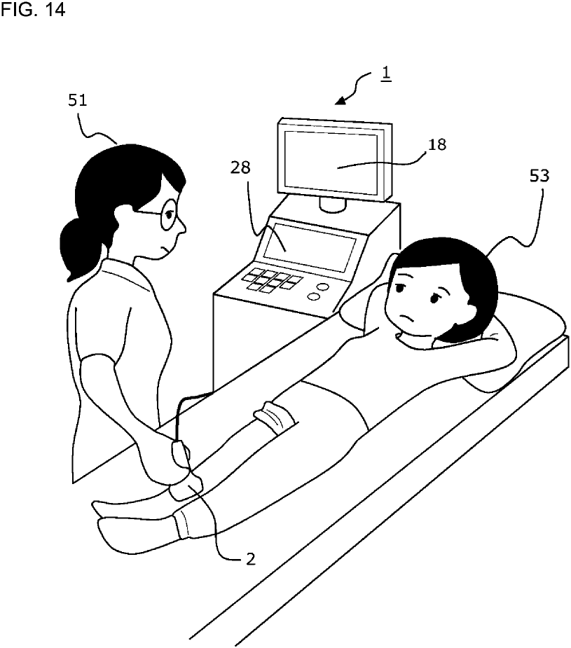

Once the examination of the subject 52 ends, the user 51 performs examination of a new subject (see FIG. 14).

FIG. 14 is a diagram illustrating an aspect of scanning a new subject 53.

A case is described below in which an examination location of the new subject 53 is different from an examination location of a subject 52 immediately prior (see FIG. 1). Here, a case is described in which the examination location of the subject 52 immediately prior is the mammary gland, yet the examination location of the new subject 53 is the lower extremities.

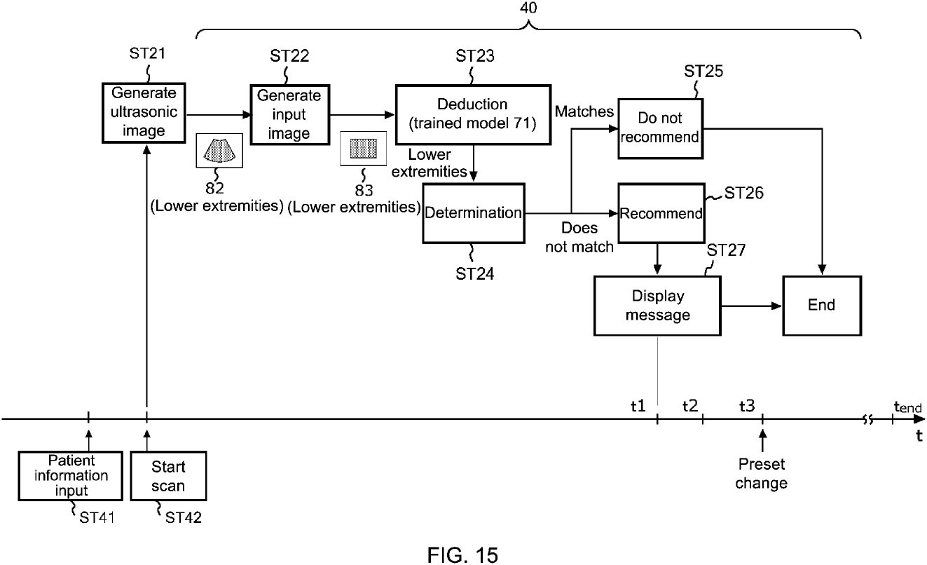

FIG. 15 is a diagram illustrating an example of a flowchart whereby the examination of the new subject 53 is executed.

In step ST41, the user 51 performs input of patient information and selection of a preset. However, when a large number of subjects must be examined, such as when there are several people awaiting examination, the user 51 may focus on starting examinations quickly and start the examination of the new subject 53 without changing the preset selected for the examination location of the subject 52 immediately prior (see FIG. 1). If the examination location of the new subject 53 is the same as the examination location of the subject 52 immediately prior, the preset selected during the examination of the subject 52 immediately prior can be used as is. Therefore, the user 51 can proceed with the examination of the new subject 53 without particular issue even without performing the work of selecting a preset.

However, the examination location of the new subject 53 may be different from the examination location of the subject 52 immediately prior. Here, as described above, the examination location of the subject 52 immediately prior is the “mammary gland”, however, consider a case in which the examination location of the new subject 53 is the “lower extremities”.

If the user 51 does not perform selection of a preset, the preset will be in the state of the preset selected for the subject 52 immediately prior in which the examination location is the “mammary gland (B0)” (see FIG. 12). Therefore, the ultrasonic diagnostic device 1 recognizes the examination location of the new subject 53 as being the “mammary gland”.

Meanwhile, since the examination location of the new subject 53 is the lower extremities, in step ST42, as illustrated in FIG. 14, the user 51 touches the ultrasonic probe 2 to the lower extremities of the new subject 53 and starts scanning.

The ultrasonic probe 2 transmits an ultrasonic wave and receives an echo reflected from within the subject 53. The received echo is converted to an electrical signal, and this electrical signal is output as an echo signal to the receiving apparatus 5. The receiving apparatus 5 executes a prescribed process on the echo signal and outputs the echo signal to the reception beamformer 6. The reception beamformer 6 executes reception beamforming on the signal received through the receiving apparatus 5 and outputs echo data.

The process next proceeds to step ST21.

In step ST21, the processor 7 generates an ultrasonic image 82 based on the echo data. The ultrasonic image 82 is an image of the lower extremities of the new subject 53.

The user 51 confirms the generated ultrasonic image 82, stores the ultrasonic image 82 as needed, and the like and continues performing work for acquiring ultrasonic images.

Meanwhile, the processor 7 executes a process 40 for determining whether to recommend a preset change to the user 51 based on the ultrasonic image 82 acquired in step ST21. The process 40 is described hereinbelow.

In step ST22, the processor 7 generates an input image 83 input to the trained model 71 based on the ultrasonic image 82.

The processor 7 executes pre-processing of the ultrasonic image 82. This pre-processing is basically the same as the pre-processing executed when generating training data for the trained model 71 (see FIG. 4). The input image 83 input to the trained model 71 can be generated by executing pre-processing. After the input image 83 is generated, the process proceeds to step ST23.

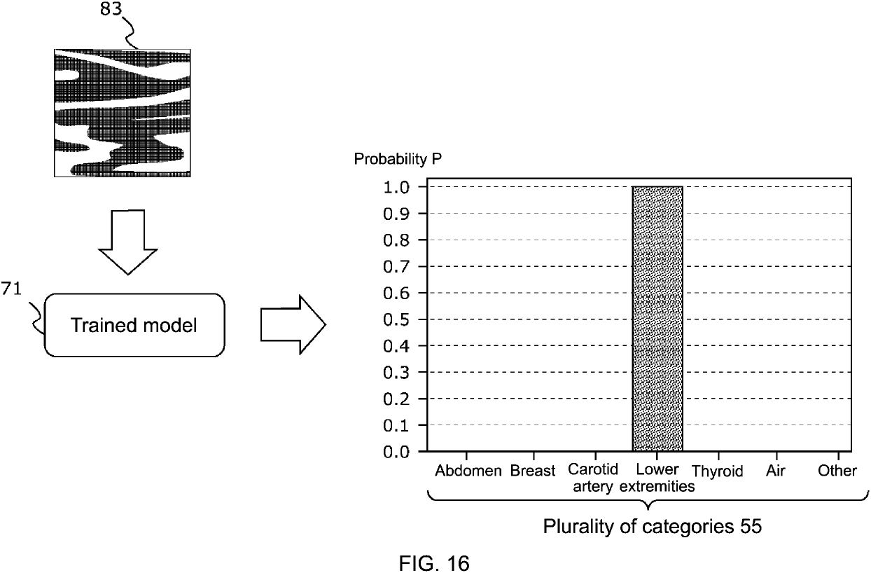

In step ST23, the processor 7 deduces a location shown by the input image 83 using the trained model 71 (see FIG. 16).

FIG. 16 is an explanatory diagram of the deduction phase of the trained model 71.

The processor 7 inputs the input image 83 to the trained model 71 and, using the trained model 71, deduces which location from among the plurality of locations of the subject is the location shown by the input image 83. Specifically, the processor 7 deduces into which category the location of the input image 83 is to be classified, selected from the plurality of categories 55 including “abdomen”, “mammary gland”, “carotid artery”, “lower extremities”, “thyroid”, “air”, and “other”. Moreover, the processor 7 obtains a probability of the location shown by the input image 83 being classified into each category.

In FIG. 16, a deduction result is output showing that the probability of the location of the input image 83 being the lower extremities is close to 100%. Therefore, the processor 7 recommends the “mammary gland” as the location illustrated by the input image 83. After deducing the location shown by the input image 81, the process proceeds to step ST24.

In step ST24, the processor 7 determines whether the examination location deduced in step ST23 matches an examination location of the preset selected by the user 51.

Here, the preset for mammary gland selected during examination of the subject 52 immediately prior is also used in the examination of the new subject 53 without being changed. Therefore, the deduced examination location is the “lower extremities”, but the selected examination location is the “mammary gland”. Therefore, in step ST24, the processor 7 determines that the examination location deduced in step ST23 does not match the examination location of the preset selected by the user 51, so the process proceeds to step ST26. In step ST26, the processor 7 determines to recommend a preset change to the user 51.

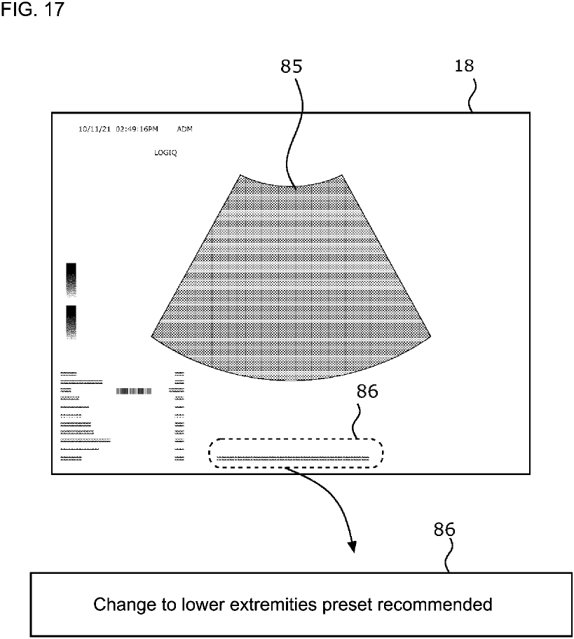

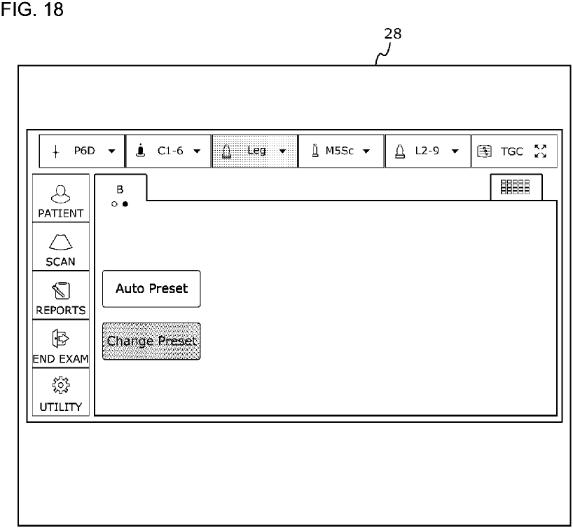

When recommending a preset change to the user 51, the processor 7 proceeds to step ST27, controls the display monitor 18 and the touch panel 28, and presents the following information to the user 51 (see FIGS. 17 and 18).

FIG. 17 is a diagram illustrating a message 86 displayed on the monitor 18, and FIG. 18 is a diagram illustrating an example of the preset change screen displayed on the touch panel 28.

The ultrasonic image 85 is displayed on the display monitor 18. In addition, the processor 7 displays the message 86 “Change to lower extremities preset is recommended” on the display monitor 18. The message 86 is for recommending that the user 51 change the preset. Seeing this message 86, the user 51 can recognize that a preset change is recommended. Note that in FIG. 17, the message 86 is displayed in a character string. However, so long as a preset change can be recommended to the user 51, the message 86 is not limited to a character string and may be, for example, a code or symbol. The message 86 may also be a combination of at least two among a character string, code, and symbol. For example, a symbol representing an examination location of a recommended preset may be displayed as the message 86. Additionally, the message 86 may be a blinking display when needed so that the user 51 realizes as quickly as possible that the message 86 is being displayed.

Moreover, as illustrated in FIG. 18, a screen for changing the preset is displayed on the touch panel 28. An “Auto Preset” button and a “Change Preset” button are displayed on the display screen. The “Change Preset” button is a button for determining whether to change a preset. When the user 51 clicks the “Change Preset” button, a signal is input indicating that the preset be changed. In response to this signal, the processor 7 can change the preset to the lower extremities preset.

On the other hand, the “Auto Preset” button is a button for determining whether an operating mode of the ultrasonic diagnostic device 1 is set to a preset change mode for automatically changing the preset. When the user 51 turns the “Auto Preset” on, the preset change mode is set. When the preset change mode is set and “Does not match” is determined in step ST24 in an examination thereafter, the message 86 is not presented to the user 51 and the preset is changed automatically. On the other hand, when the preset change mode is turned off, the operating mode of the ultrasonic diagnostic device 1 stays in a preset recommendation mode selected by the user 51 selecting a preset.

Thus, during an examination, the user 51 can change a preset, set an operating mode of the ultrasonic diagnostic device 1 to the preset change mode, and the like such that the settings suit a preference of the user 51.

Note that in FIG. 15, a time “t1” when the message 86 is displayed is shown. In the middle of examining the new subject 53, the user 51 notices the message 86 (see FIG. 17) displayed on the display monitor 18. In FIG. 15, a time “t2” when the user 51 noticed the message 86 is shown. By seeing the message 86, the user 51 notices that the currently selected preset is not the preset for the lower extremities.

Therefore, by turning the “Change Preset” button on the preset change screen displayed on the touch panel 28 on (see FIG. 18), the user 51 can change the preset for the mammary gland, which is currently selected, to the preset for the lower extremities, being the examination location of the subject 53.

Meanwhile, even when the user 51 notices the message 86, there may be cases where, due to progress of the examination, progress of work of the user 51, or the like, the user does not change the preset immediately, and then decides later to change the preset. For example, conceivable cases include a case that ends with just a cross-section scan when an ultrasonic image from a cross-sectional scan currently being worked seems to be of satisfactory quality, but then after this scan has ended, a decision is made that a preset change is desired, and a case of completing work first due to the work currently being worked on being high priority then deciding after the scan has ended that a preset change is desired. In such cases, the user 51 can change the preset at a convenient time, rather than immediately changing the preset as soon as the message 86 is noticed by the user 51.

Therefore, the user 51 can change the preset at a time convenient for proceeding with the examination of the new subject 53 rather than immediately changing the preset at a time t2 when the message 86 is noticed. For example, the user 51 can change the preset at a time t3 when prescribed work has settled down without immediately changing the preset at the time t2 when the message 86 is noticed.

Once the preset is changed, the user 51 restarts the examination and when ultrasonic images necessary for diagnosis are acquired, the examination is ended.

In Embodiment 1, when an examination location set by a preset is different than a deduced examination location, the processor 7 controls the display monitor 18 such that the message 86 “Change to lower extremities preset is recommended” is displayed on the display monitor 18. Seeing the message 86, the user 51 is able to notice, in the middle of performing work to scan the subject 53 while operating the ultrasonic probe 2, that the preset currently selected is not the lower extremities preset. Therefore, the user 51 is able to change the preset on the preset change screen (see FIG. 18).

Moreover, even when the user 51 notices the message 86, there may be cases where the user does not change the preset immediately, then decides later that they want to change the preset due to progress of the examination, progress of work of the user 51, or the like. In such cases, since the user 51 is able to change the preset after their high priority work is completed, the user 51 can change the preset at a convenient time rather than immediately changing the preset as soon as the message 86 is noticed by the user 51.

In addition, in Embodiment 1, when an examination location selected by the user 51 is different than a deduced examination location, the message “Change to lower extremities preset is recommended” is displayed on the display monitor 18 without the preset being changed compulsorily. Therefore, the risk of the image quality of the ultrasonic image conversely being worse due to the preset being changed automatically can be avoided when there is a low possibility of a deduced examination location matching an actual examination location of a subject.

Note that in Embodiment 1, the process of deducing an examination location is only executed once during an examination of the subject 53, however, the process of deducing an examination location may be executed repeatedly while the examination of the subject 53 is being performed. For example, the user 51 may need to examine a plurality of examination locations of the subject 53 in one examination, and in this case, may want to change a preset for each examination location of the subject 53. Therefore when, after an examination of a given examination location of the subject 53 has ended, and examination of a separate examination location of the subject 53 is started without changing the preset, a process of deducing the examination location may be executed repeatedly while the examination of the subject 53 is being performed so that a preset change can be recommended to the user 51.

In addition, in Embodiment 1, examination location is deduced in step ST23, however, when the probability P of the deduced examination location is low (for example, 60% or lower), the reliability of the deduction results drops and there is a risk that a preset for an examination location that is different from the actual examination location of the subject 53 will be recommended to the user. Therefore, in order to avoid such risk, when the probability P is low, it is desirable that the process 40 be ended without a preset change being recommended to an operator.

### Embodiment 2

In Embodiment 2, an example is described in which, after displaying the message 86, the processor 7 determines whether a user has executed a prescribed operation, and changes a preset when it is determined that the user has executed the prescribed operation.

In Embodiment 2, similarly to Embodiment 1, an examination of the subject 52 is performed in which the examination location is the mammary gland, then examination of the new subject 53 is performed in which the examination location is the lower extremities.

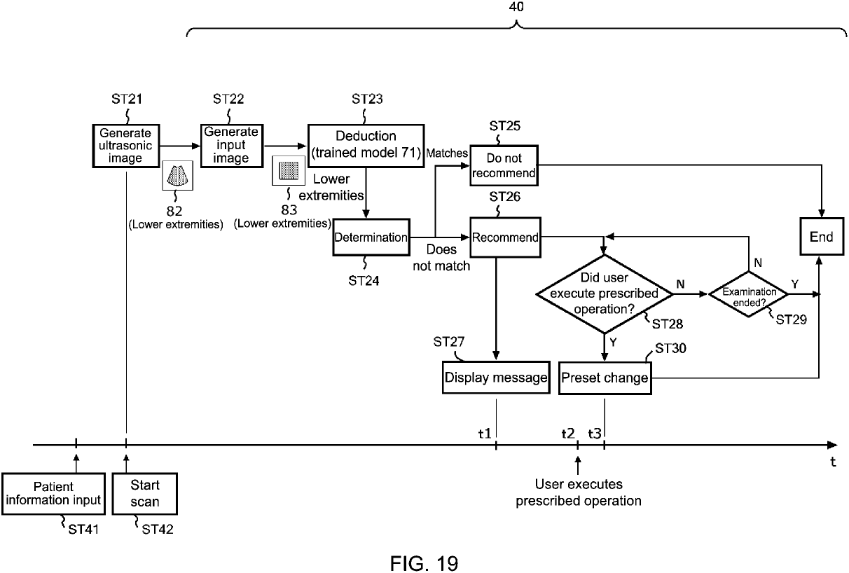

Note that the flow of examination of the subject 52 is the same as the flow described referencing FIG. 8, and therefore the flow of examination of the subject 52 is omitted, and a flow of examination of the new subject 53 in which the examination location is the lower extremities is described with reference to FIG. 19.

FIG. 19 is a diagram illustrating a flow of examination of the new subject 53 in Embodiment 2.

Note that steps ST41, ST42, and ST21 to ST27 are the same as steps ST41, ST42, and ST21 to ST27 described referencing FIG. 15. As such, descriptions thereof are omitted.

In step ST23, after the processor 7 deduces an examination location of the subject 53, the process proceeds to step ST24.

In step ST24, the processor 7 determines whether the examination location deduced in step ST23 matches an examination location of the preset selected by the user 51.

Here, the preset for mammary gland selected during an examination of the subject 52 immediately prior is also used in the examination of the new subject 53 without being changed. Therefore, the deduced examination location is the “lower extremities”, but the selected examination location is the “mammary gland”. Therefore, in step ST24, the processor 7 determines that the examination location deduced in step ST23 does not match the examination location of the preset selected by the user 51 and therefore proceeds to step ST26, whereupon the processor 7 makes a determination to recommend a preset change to the user 51.

When recommending a preset change to the user 51, the processor 7 proceeds to step ST27 and displays the message 86 “Change to lower extremities preset is recommended” on the display monitor 18 (see FIG. 17).

Seeing the message 86, the user 51 can notice that a currently selected preset is not the lower extremities preset. However, it is conceivable here that, due to the user 51 working on work of higher priority than changing a preset setting, the user 51 may not change the preset immediately.

In this case, the processor 7 proceeds to step ST28 and determines whether the user 51 executed prescribed operations for interrupting transmission and reception of ultrasonic waves. It is believed that work of the user will not be adversely affected when transmission and reception of the ultrasonic waves is interrupted even when a preset is changed, so in Embodiment 2, the user 51 changes a preset when the prescribed operations for interrupting transmission and reception of ultrasonic waves have been executed. Here, the prescribed operations are, for example, a freeze operation, a screen storing operation, and a depth change operation.

In step ST28, when the processor 7 determines whether the prescribed operations for interrupting transmission and reception of ultrasonic waves were executed or not, and determines that the prescribed operations were not performed, the process proceeds to step ST29. In step ST29, a determination is made as to whether the examination has ended. When the examination is completed, the process 40 terminates. On the other hand, if it is determined in step ST29 that the examination has not ended, the process proceeds to step ST28. Therefore, a looped repetition of steps ST28 and ST29 is executed until it is determined, in step ST28, that the user 51 executed the prescribed operations or it is determined, in step ST29, that the examination has ended.

In Embodiment 2, the user 51 performs a prescribed operation at the time t2, which is a certain amount of time after the time t1 when the message 86 is displayed. Therefore, at the time t2, reception and transmission of the ultrasonic waves are interrupted by the prescribed operations of the user 51. In this case, in step ST28, the processor 7 determines that the user 51 executed the prescribed operations. The process then proceeds to step ST30. In step ST30, the processor 7 changes the mammary gland preset to the lower extremities preset. In FIG. 19, the time when the preset is changed is shown as “t3”. When the preset is changed, the processor 7 can display a message on the display monitor 18 (or the touch panel 28) informing the user 51 that the preset was changed. Seeing this message, the user 51 can recognize that the preset was changed. After the prescribed operations are executed by the user 51, the user 51 can continue the examination of the subject 53 using a preset for the actual examination location of the subject 53.

In Embodiment 2, when transmission and reception of ultrasonic waves are interrupted by the prescribed operations of the user, the selected preset can be automatically changed to a preset of a deduced examination location. As such, the user 51 can continue the examination of the subject 53 using the preset for the actual examination location of the subject 53 even without the user 51 changing the preset.

### Embodiment 3

In Embodiment 3, an example is given in which the probability P and two thresholds TH1 and TH2 are compared and it is determined based on the result of this comparison whether to recommend a preset change to the user 51.

In Embodiment 3, similarly to Embodiment 1, an examination of the subject 52 is performed in which the examination location is the mammary gland, then examination of the new subject 53 is performed in which the examination location is the lower extremities.

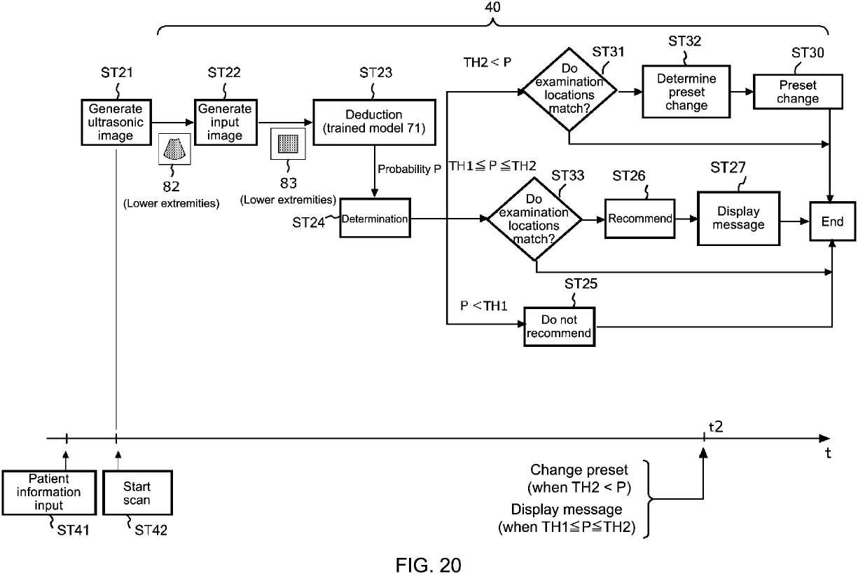

Note that the flow of examination of the subject 52 is the same as the flow described referencing FIG. 8, so the flow of examination of the subject 52 is omitted and a flow of examination of the new subject 53 in which the examination location is the lower extremities is described with reference to FIG. 20.

FIG. 20 is a diagram illustrating a flow of examination of the new subject 53 in Embodiment 3.

Note that steps ST41, ST42, and ST21 to ST23 are the same as steps ST41, ST42, and ST21 to ST23 described referencing FIG. 15. As such, a description thereof is omitted.

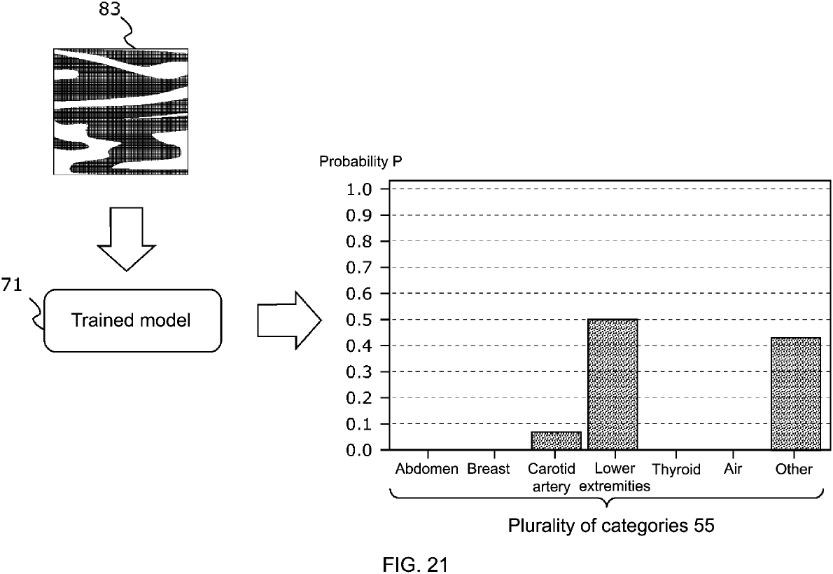

In step ST23, the processor 7 deduces an examination location of the subject 53 (see FIG. 21).

FIG. 21 is an explanatory diagram of the deduction phase of the trained model 71.

The processor 7 inputs the input image 83 to the trained model 71 and, using the trained model 71, deduces which location from among the plurality of locations of the subject is the location shown by the input image 83. Specifically, the processor 7 deduces into which category the location shown by the input image 83 is to be classified, selected from the plurality of categories 55 including “abdomen”, “mammary gland”, “carotid artery”, “lower extremities”, “thyroid”, “air”, and “other”. Moreover, the processor 7 obtains a probability of the location shown by the input image 83 being classified into each category.

In FIG. 21, the processor 7 deduces that the location shown by the input image 81 is classified as “carotid artery”, “lower extremities”, and “other”. Moreover, the processor 7 determines the probability P of the location shown by the input image 81 being classified as “carotid artery” to be 8%, the probability of being classified as “lower extremities” to be 50%, and the probability P of being classified as “other” to be 42%. Therefore, the probability P for the lower extremities is highest at P = 50%, so the processor 7 deduces that the location of the input image 83 is the lower extremities.

In step ST23, once a deduction result is output, the process proceeds to step ST24.

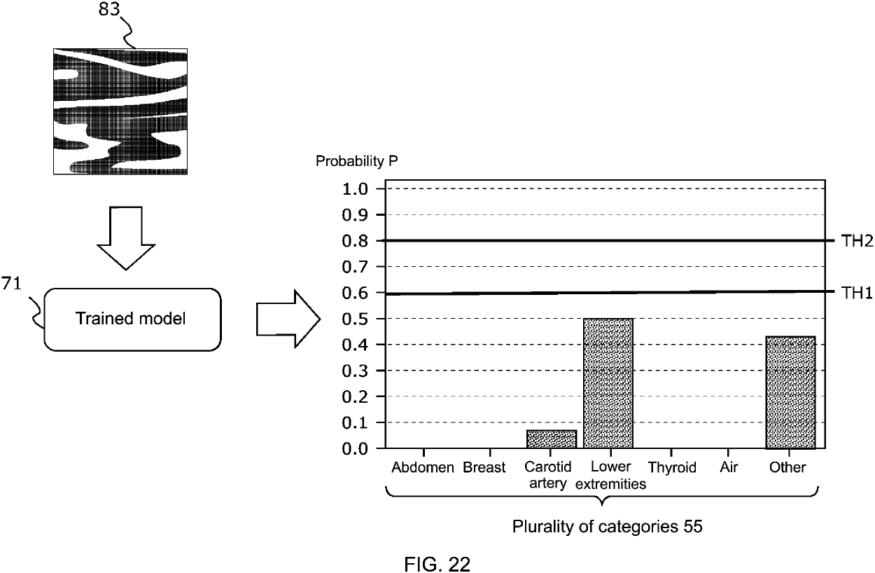

FIG. 22 is an explanatory diagram of the process in step ST24.

The processor 7 compares the probability P (= 50%) for the deduced “lower extremities” and the two thresholds TH1 and TH2 to determine whether the probability P is lower than the threshold TH1 (P < TH1), whether the probability P is a value between the thresholds TH1 and TH2 (TH1 ≦ P ≦ TH2), or whether the probability P is greater than the threshold TH2 (TH2 < P).

In Embodiment 3, the processor 7 determines whether to present the message 86 according to the range (P < TH1, TH1 ≦ P ≦ TH2, TH2 < P) in which the probability P is included. Therefore, operations of the processor 7 are described below for when the probability P is sorted into P < TH1, when sorted into TH1 ≦ P ≦ TH2, and when sorted into TH2 < P.

### When P < TH1

As previously described, referencing the deduction results shown in FIG. 22, the probability that the input image 83 is the lower extremities is 50%. Therefore, since the probability P for the lower extremities is lower than the first threshold TH1, the probability P is a value within the range P < TH1.

The first threshold TH1 is a standard value indicating the probability that the location shown by the input image 83 and the deduced examination location match is low. Here, the first threshold TH1 is set to 60(%) but may be set to a different value. Since the first threshold TH1 is a standard value indicating the probability that the location shown by the input image 83 and the deduced examination location match is low, the probability that the location shown by the input image 83 and the deduced examination location match is considered low when the probability P is lower than the first threshold TH1. Therefore, if a preset is changed when the probability P is lower than the first threshold TH1, there is a risk that examination will be performed using a preset for an examination location different from an actual examination location of the subject 53. Thus, in order to avoid performing examination of the subject 53 using a preset for an examination location different from an actual examination location of the subject 53, when the probability P is within the range P < TH1, the processor 7 proceeds to step ST25 (see FIG. 20) and determines not to recommend a preset change to the user 51, and the process 40 ends. As such, no preset change is recommended when the probability P is lower than the first threshold TH1, so the risk of the image quality of the ultrasonic image being worse due to the preset being changed by the user 51 can be avoided.

### When TH2 < P

Next, a case is described for the probability P when TH2 < P.

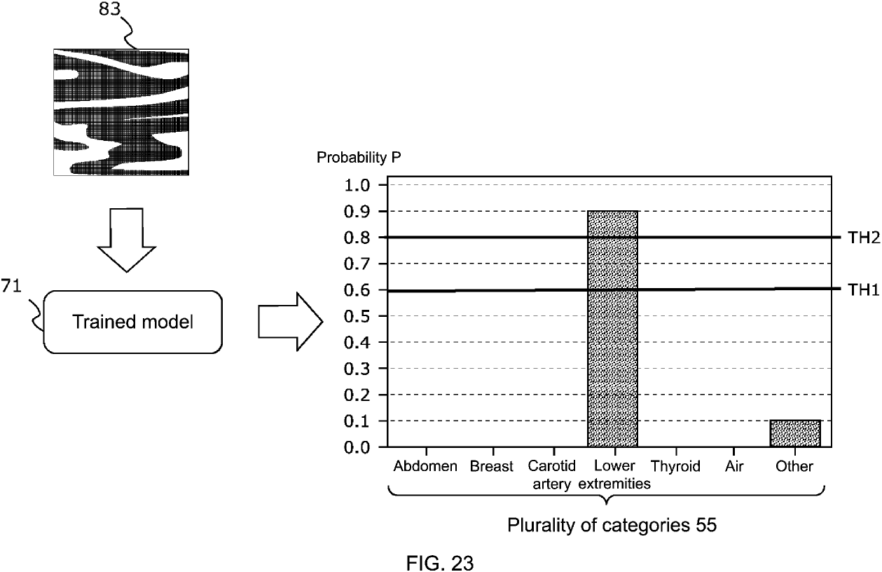

In step ST23, the processor 7 deduces an examination location of the subject 53. FIG. 23 is a diagram illustrating an example of deduction results when the probability P is TH2 < P. Referring to the deduction results, in FIG. 23, the probability that the input image 83 is the lower extremities is 90%, and the probability that the input image 83 is “other” is 10%. Therefore, the probability P for the lower extremities is highest at P = 90%, so the processor deduces that the location of the input image 83 is the lower extremities, and the process proceeds to step ST24.

In step ST24, the processor 7 compares the probability P (= 90%) for the deduced “lower extremities” and the two thresholds TH1 and TH2 to determine whether the probability P is lower than the threshold TH1 (P < TH1), whether the probability P is a value between the thresholds TH1 and TH2 (TH1 ≦ P ≦ TH2), or whether the probability P is greater than the threshold TH2 (TH2 < P).

Referencing the deduction results shown in FIG. 23, the probability that the input image 83 is the lower extremities is 90%. Therefore, the probability P is greater than the threshold TH2 (TH2 < P), so the process proceeds to step ST31.

In step ST31, the processor 7 determines whether the deduced examination location matches an examination location of a preset selected during examination of the previous subject 52. No preset change is necessary when the examination locations match, so it is determined not to change the preset and the flow ends.

On the other hand, when the examination locations do not match, the process proceeds to step ST32. Here, the preset selected during examination of the previous subject 52 is also used in the examination of the new subject 53 without being changed. Therefore, the deduced examination location is the “lower extremities”, but the selected examination location is the “mammary gland”. Therefore, the processor 7 determines that the deduced examination location does not match the examination location of the preset selected by the user 51, so the process proceeds to step ST32.

In step ST32, the processor 7 determines to change the preset automatically without recommending a preset change to the user 51. The reason why no preset change is recommended is described below.

As illustrated in FIG. 23, the probability P has a value greater than the second threshold TH2 (TH2 < P). The second threshold TH2 is a value greater than the first threshold TH1, being a standard value indicating the probability that the location shown by the input image 83 and the deduced examination location match to be high. Here, the second threshold TH2 is set to 80(%) but may be set to a different value. As described above, since the second threshold TH2 is a standard value indicating the probability that the location shown by the input image 83 and the deduced examination location match to be high, the possibility that the location shown by the input image 83 and the deduced examination location match is considered to be extremely high when the probability P is greater than the second threshold TH2. Therefore, it is believed that having the preset change automatically rather than having the user 51 perform the work of changing a preset can reduce a work load on the user 51 while maintaining examinations of satisfactory quality. Thus, when the probability P is greater than the second threshold TH2, in step ST32, the processor 7 determines to change the preset without recommending a preset change to the user 51. When it is determined that a preset be changed, the processor 7 proceeds to step ST30 and automatically changes a selected preset to a preset for a deduced examination location. The processor 7 changes the preset at the time t2, for example. Once the preset has changed, the process 40 ends.

When the probability P is high, automatically changing the preset enables a reduced work load on the user 51 while maintaining examinations of satisfactory quality.

### When TH1 ≤ P ≤ TH2

Finally, the case is described for the probability where TH1 ≤ P ≤ TH2.

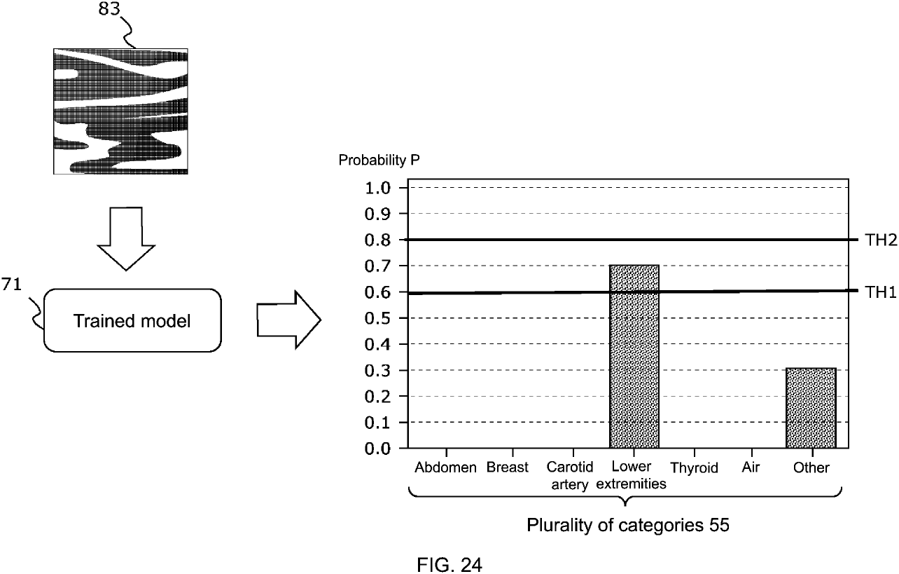

In step ST23, the processor 7 deduces an examination location of the subject 53. FIG. 24 is a diagram illustrating an example of a deduction result for the probability P when TH1 ≦ P ≦ TH2. Referring to the deduction results, in FIG. 24, the probability of the input image 83 being classified as lower extremities is 70%, and the probability of the location of the input image 83 being classified as “other” is 30%. Therefore, the probability P for the lower extremities is highest at P = 70%, so the processor deduces that the location of the input image 83 is the lower extremities, and the process proceeds to step ST24.

In step ST24, the processor 7 compares the probability P (= 70%) for the deduced “lower extremities” and the two thresholds TH1 and TH2 to determine whether the probability P is lower than the threshold TH1 (P < TH1), whether the probability P is a value between the thresholds TH1 and TH2 (TH1 ≦ P ≦ TH2), or whether the probability P is greater than the threshold TH2 (TH2 < P).

Referencing the deduction results shown in FIG. 24, the probability of the input image 83 being classified as lower extremities is 70%. Therefore, the probability P is within the range TH1 ≦ P ≦ TH2, so the process proceeds to step ST33.

In step ST33, the processor 7 determines whether the deduced examination location matches an examination location of a preset selected during examination of the previous subject 52. No preset change is necessary when the examination locations match, so it is determined not to change the preset and the flow ends.

On the other hand, when the examination locations do not match, the process proceeds to step ST26. Here, the preset set during examination of the previous subject 52 is also used in the examination of the new subject 53 without being changed. Therefore, the deduced examination location is the “lower extremities”, but the selected examination location is the “mammary gland”. Therefore, the processor 7 determines that the deduced examination location does not match the examination location of the preset selected by the user 51, so the process proceeds to step ST26.

In step ST26, the processor 7 determines to recommend a preset change to the user 51. Therefore, the case for the probability P when TH1 ≦ P ≦ TH2 differs from the case for the probability P when TH2 < P. A preset change is recommended to the user 51 without a preset being changed automatically. The reason why the preset change is recommended to the user 51 without the preset being changed automatically is described below.

As illustrated in FIG. 24, the probability P has a value between the first threshold TH1 and the second threshold TH2 (TH1 ≦ P ≦ TH2). As such, the probability P is considered neither high nor low. In such a case, it is believed that deferring the determination to the user 51 as to whether to change a preset rather than changing the preset compulsorily enables a correct preset to be reliably selected. Therefore, when the probability P is within the first threshold TH1 and second threshold TH2, in step ST26, the processor 7 determines to recommend a preset change to the user 51 without changing the preset automatically. The processor 7 proceeds to step ST27 and displays the message 86 (see FIG. 17) for recommending the preset change to the user 51 on the display monitor 18. The processor 7 displays the message 86 at the time t2, for example. Once the message 86 is displayed, the process 40 ends.

The user 51 can change the preset at a convenient time for the user 51 after noticing the message 86, which enables efficient examination of the subject 53 to be performed.

Note that when TH1 ≦ P ≦ TH2, steps ST28 and ST30 shown in FIG. 19 for Embodiment 2 (changing a preset in response to prescribed operations by the user 51) may be executed.

### Embodiment 4

In Embodiment 4, an example is described in which deduction of an examination location is stopped as needed.

In Embodiment 4, similarly to Embodiment 1, an examination of the subject 52 is performed in which the examination location is the mammary gland, then examination of the new subject 53 is performed in which the examination location is the lower extremities.

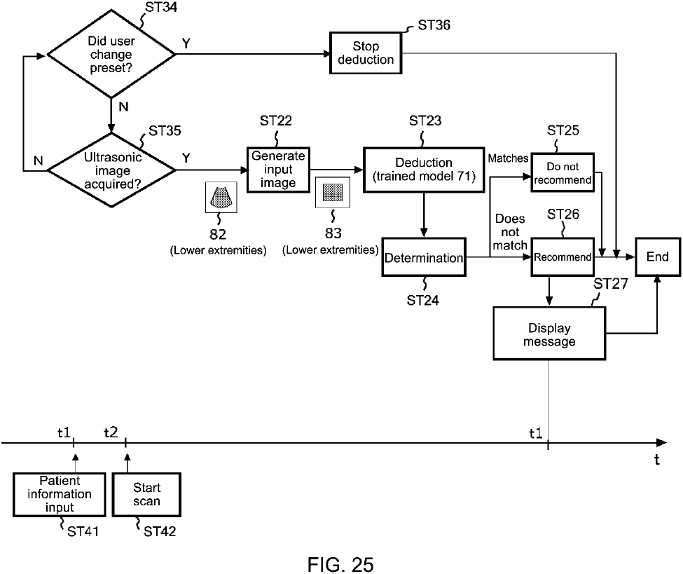

Note that the flow of examination of the subject 52 is the same as the flow described referencing FIG. 8, so the flow of examination of the subject 52 is omitted and a flow of examination of the new subject 53 in which the examination location is the lower extremities is described with reference to FIG. 25.

FIG. 25 is a diagram illustrating a flow of examination of the new subject 53 in Embodiment 4.

In step ST41, the user 51 leads the new subject 53 to an examination room and has the subject lie down on an examination bed. In addition, the user 51 operates the user interface 10 to set each of an item that must be set in advance before scanning the new subject 53. For example, the user 51 operates the user interface 10 to input patient information.

As illustrated in FIG. 9, the user 51 displays a settings screen for patient information on the touch panel 28. Once the settings screen is displayed, the user clicks a “new patient” button 31. By this button 31 being clicked, an input screen 32 for patient information is displayed. The user 51 inputs the patient information and other information as needed. For example, when the user 51 clicks the “New patient” button 31 or when input of required patient information is completed, the processor 7 can determine whether a signal indicating the start of the examination of the subject 52 was input. Therefore, for example, the ultrasonic diagnostic device 1 can recognize that the examination of the subject 52 has started by the user 51 clicking the “new patient” button 31.

When examination of the new subject 53 is started, in step ST34, the processor 7 determines whether the user 51 changed a preset. When an examination location of the new subject 53 is different from an examination location of the subject 52 immediately prior, generally, the user 51 performs the examination after changing the preset. Therefore, the preset being changed after the start of examination of the new subject 53 is considered a change by the user 51 of the preset selected during examination of the previous subject 52 (see FIG. 1) to a preset for the examination location of the new subject 53. In this case, the preset for the examination location of the new subject 53 is selected intentionally by the user 51, so recommending a preset change to the user 51 is considered unnecessary. Therefore, in step ST34, when the processor 7 determines whether a preset was changed by the user 51 and it is determined that the preset was changed by the user 51, the processor proceeds to step ST36, stops the deduction of the examination location, and ends the flow. In this case, the user 51 performs examination of the subject 53 according to presets set by the user 51.

On the other hand, when it is determined that the user 51 did not change the preset, the processor proceeds to step ST35. In step ST35, it is determined whether an ultrasonic image was acquired. When it is determined that an ultrasonic image has not yet been obtained, the processor returns to step ST34 and it is determined whether the user 51 changed the preset. Therefore, steps ST34 and ST35 are looped repeatedly until it is determined, in step ST34, that the user 51 changed a preset or it is determined, in step ST35, that an ultrasonic image was acquired. When the ultrasonic image 82 is acquired, the processor proceeds to step ST22 and, similarly to Embodiment 1, executes a process for recommending a preset change to the user 51.

In ultrasonic examinations, there are cases, in addition to when acquiring ultrasonic images of a plurality of examination locations in a single examination, when an ultrasonic image of one examination location is acquired in a single examination. In the latter case, only the ultrasonic image of the one examination location is acquired in the single examination, so after changing a preset, the user 51 must reselect a new preset until the examination has ended. However, deduction (step ST23) is executed after the user 51 has selected a preset for an examination location of the subject 53, and when, as a result, a location separate from an actual examination location is deduced as the examination location, there is a risk that recommending a preset change to the user 51 may conversely lead to reduced image quality. Therefore, in order to avoid such a risk, in an examination in which only an ultrasonic image of one examination location is acquired, when the user 51 has changed the preset, it is determined that no new preset needs to be reselected until the examination has ended, and in step ST36, the processor 7 stops deduction. As such, in Embodiment 4, since performance of deduction is stopped after the user 51 has changed the preset, the risk of execution of unnecessary deduction conversely leading to reduced image quality can be avoided.

Note that step 36 for stopping deduction may also be applied to other Embodiments, for example, Embodiment 3 and the like.

### Embodiment 5

In Embodiment 5, an example of the deduction executed in Embodiment 4 is described in which deduction is stopped using different timing.

In Embodiment 5, similarly to Embodiment 1, an examination of the subject 52 is performed in which the examination location is the mammary gland, then examination of the subject 53 is performed in which the examination location is the lower extremities.

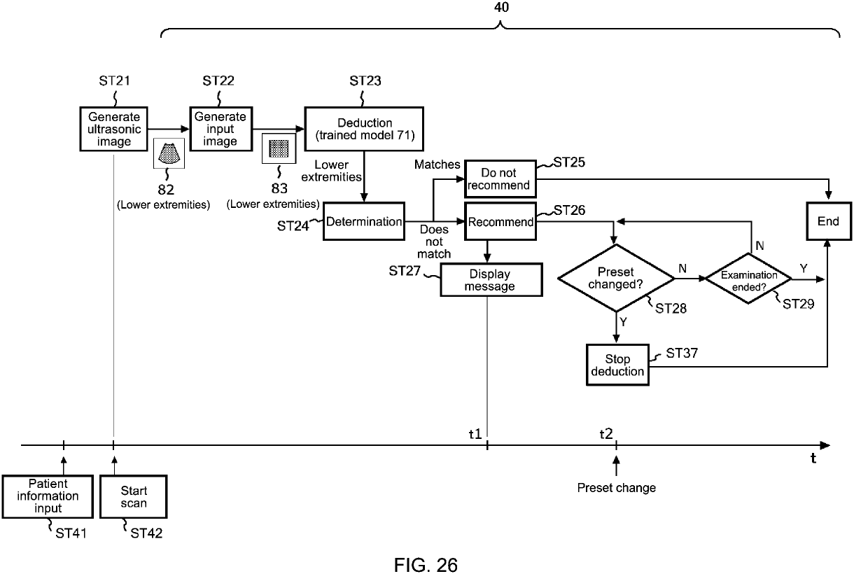

Note that the flow of examination of the subject 52 is the same as the flow described referencing FIG. 8, so the flow of examination of the subject 52 is omitted and a flow of examination of the new subject 53 in which the examination location is the lower extremities is described with reference to FIG. 26.

FIG. 26 is a diagram illustrating a flow of examination of the new subject 53 in Embodiment 5.

Note that comparing the flow in FIG. 26 for Embodiment 5 to the flow in FIG. 15 for Embodiment 1, the two differ in the addition of steps ST28, ST29, and ST37, but are otherwise the same as the flow in FIG. 15. Therefore, primarily steps ST28, ST29, and ST37 are described below while the other steps are described briefly.

In step ST23, after the processor 7 deduces an examination location of the new subject 53, the process proceeds to step ST24.

In step ST24, the processor 7 determines whether the examination location deduced in step ST23 matches an examination location of the preset selected by the user 51.

Here, the preset for the mammary gland set during an examination of the subject 52 immediately prior is also used in the examination of the new subject 53 without being changed. Therefore, the deduced examination location is the “lower extremities”, but the selected examination location is the “mammary gland”. Therefore, in step ST24, the processor 7 determines that the examination location deduced in step ST23 does not match the examination location of the preset selected by the user 51 and therefore proceeds to step ST26, whereupon the processor 7 makes a determination to recommend a preset change to the user 51.

When recommending a preset change to the user 51, the processor 7 proceeds to step ST27 and displays the message 86 “Change to lower extremities preset is recommended” on the display monitor 18 (see FIG. 17).

In addition, after recommending the preset change to the user 51, the processor 7 proceeds to step ST28. In step ST28, the processor 7 determines whether the preset was changed by the user 51.

Meanwhile, once the user 51 notices the message 86 “Change to lower extremities preset is recommended” displayed at the time t1 on the display monitor 18, the user 51 changes the preset at a convenient time. In FIG. 26, the time when the user 51 changed the preset is shown as “t2”.

When the user 51 changed the preset, in step ST28, the processor 7 determines that the preset was changed by the user 51 and proceeds to step ST37. In step ST37, the processor 7 stops deduction and ends the process 40.

In Embodiment 5, deduction is executed in step ST23, and based on results of this deduction, the message 86 “Change to lower extremities preset is recommended” is displayed on the display monitor 18 (time t1). The user 51 follows the recommendation of this message 86, changes the preset at the time t2, and continues examining the subject. Thus, based on the intentions of the user 51, the user 51 changes the preset for the examination location such that the preset corresponds to an actual examination location of the subject 53. Therefore, in an examination in which only an ultrasonic image of one examination location is acquired, after changing the preset, the user 51 does not need to select a new preset until the examination has ended. Meanwhile, a second round of deduction is executed regardless of whether the preset for the examination location of the subject 53 is changed by the user 51, and when, as a result, a location separate from the actual examination location is deduced as the examination location, there is a risk that recommending a preset change to the user 51 may conversely lead to reduced image quality. Therefore, in order to avoid such a risk, in an examination in which only an ultrasonic image of one examination location is acquired, when the user 51 has followed the recommendation of the message 86 and changed the preset, it is determined that no new preset needs to be reselected until the examination has ended, and in step ST37, the processor 7 stops deduction. As such, in Embodiment 5, since performance of deduction is stopped after the user 51 has changed the preset at the time t2, the risk of execution of unnecessary deduction conversely leading to reduced image quality can be avoided.

Note that step 37 for stopping deduction may also be applied to other Embodiments, for example, Embodiment 3 and the like.

### Embodiment 6

In Embodiment 6, an example is described in which deduction is stopped after a preset is changed automatically.

In Embodiment 6, similarly to Embodiment 1, an examination of the subject 52 is performed in which the examination location is the mammary gland, then examination of the subject 53 is performed in which the examination location is the lower extremities.

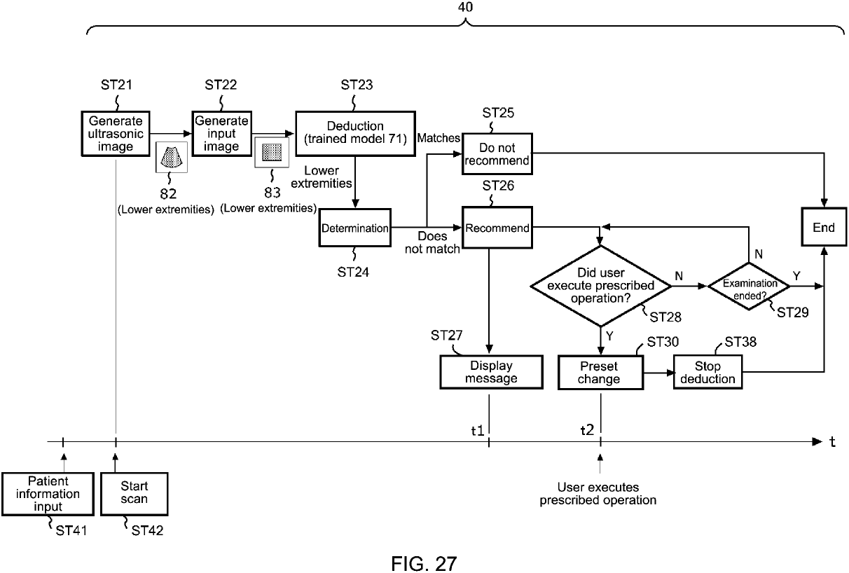

Note that the flow of examination of the subject 52 is the same as the flow described referencing FIG. 8, so the flow of examination of the subject 52 is omitted and a flow of examination of the new subject 53 in which the examination location is the lower extremities is described with reference to FIG. 27.

FIG. 27 is a diagram illustrating a flow of examination of the new subject 53 in Embodiment 6.

Note that comparing the flow in FIG. 27 for Embodiment 6 to the flow in FIG. 19 for Embodiment 2, the two differ in the addition of step ST38, but are otherwise the same as the flow in FIG. 19. Therefore, primarily step ST38 is described below while the other steps are described briefly.

In step ST26, once it is determined to recommend a preset change to the user 51, the processor 7 proceeds to step ST27. In step ST27, the processor 7 displays the message 86 “Change to lower extremities preset is recommended” on the display monitor 18 (see FIG. 17).

In addition, the processor 7 proceeds to step ST28 and determines whether the user 51 executed prescribed operations for interrupting transmission and reception of ultrasonic waves. It is believed that work of the user will not be adversely affected when transmission and reception of ultrasonic waves is interrupted even when a preset is changed. Therefore, when the user 51 executes a prescribed operation for interrupting transmission and reception of ultrasonic waves, the preset changes as described in Embodiment 2. Here, the prescribed operations are, for example, a freeze operation, a screen storing operation, and a depth change operation.

In FIG. 27, the user 51 performs the prescribed operation at the time t2. Thus, when the user 51 has performed the prescribed operation at the time t2, in step ST28, the processor 7 determines that the user 51 executed the prescribed operation and proceeds to step ST30. In step ST30, in response to the prescribed operation of the user 51, the processor 7 changes the mammary gland preset to the lower extremities preset. After the preset is changed, the processor 7 proceeds to step ST38 and stops deduction.

In Embodiment 6, after a preset is changed automatically by the processor 7 in response to an operation of the user, the second round of deduction is stopped from being performed. Thus, the risk of executing unnecessary deduction conversely leading to reduced image quality can be avoided.

Note that step 38 for stopping deduction may also be applied to other Embodiments, for example, Embodiment 3 and the like.

### Embodiment 7

In Embodiment 7, an example is described in which a probability P of a particular examination location is weighted.

As described in Embodiments 1 to 6, in step ST23, the trained model 71 is used to obtain the probability P of the location of the input image being classified into each category (for example, see FIG. 21).

The trained model 71 is created through learning of training data prepared for each examination location. However, there may also be different amounts of training data between the examination locations that can be prepared. For example, a large amount of training data may be prepared for a given examination location, whereas an amount of training data that may be prepared for another examination location may be small. Also, characteristics of the training data may differ between examination locations. As a result, the probability P for a particular examination location may be deduced low due to differences in the training data between the examination locations and/or differences in the characteristics of the training data, and the like.

Thus, the probability P of the particular examination location may be weighted. For example, in FIG. 21, the probability P deduced for the carotid artery is 5%, but a process of boosting the probability P from 5% to 10%, for example, may be executed.

### Embodiment 8

In Embodiment 8, an example in which the deduction results of step ST23 are displayed on the display 18 is described.

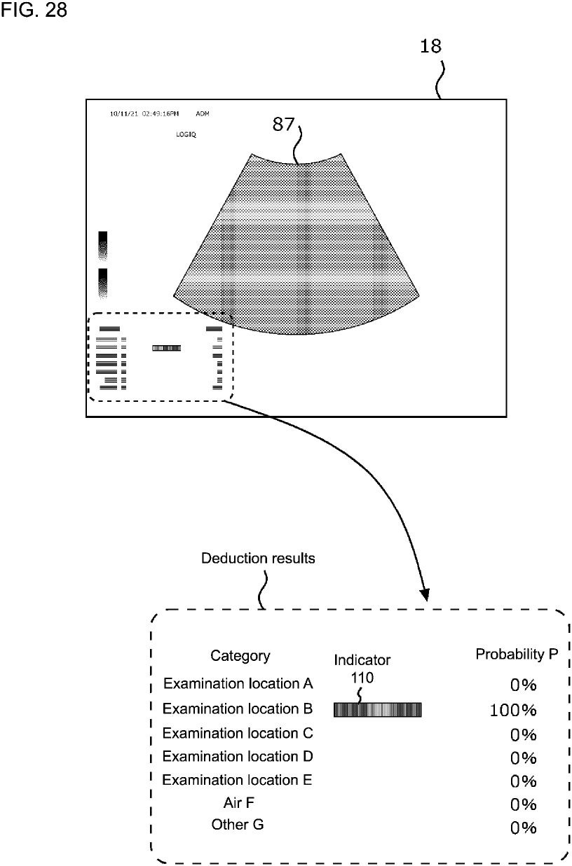

FIG. 28 is a diagram illustrating an example of deduction results displayed on a display monitor 18.

An ultrasonic image 87 used to obtain the probability P is shown on the display monitor 18.

The deduction results are displayed at the bottom left of the screen.

The deduction results include a column indicating a category, a column indicating an indicator, and a column indicating probability.

Here, for convenience of explanation, the categories are represented by examination locations A, B, C, D, and E, air F, and other G. The examination locations A, B, C, D, and E are, for example, abdomen, mammary gland, carotid artery, lower extremities, and thyroid, but may be other examination locations.

The probability P indicates a probability (for example, see FIGS. 21 to 24) of a location shown by the input image 83 being classified into each category.

In addition, an indicator 110 corresponding to a probability value is displayed between the category and the probability P.

The processor 7 determines a color of the indicator 110 based on the probability P and the thresholds TH1 and TH2 (See FIGS. 21 to 24). Specifically, the processor 7 determines the color of the indicator 110 based on whether the probability P is lower than the threshold TH1 (P < TH1), whether the probability P is a value between the thresholds TH1 and TH2 (TH1 ≦ P ≦ TH2), or whether the probability P is greater than the threshold TH2 (TH2 < P). For example, when the probability P is determined to be lower than the threshold TH1 (P < TH1), the color of the indicator 110 is determined to be red, when the probability P is greater than the threshold TH2 (TH2 < P), the color of the indicator 110 is determined to be green, and when the probability P is between the threshold TH1 and the threshold TH2 (TH1 ≦ P ≦ TH2), the indicator 110 is determined to be yellow.

In FIG. 28, the probability P is 100% and the indicator 110 is displayed green.

Therefore, by viewing the deduction results, the user 51 can visually recognize which location of the subject 53 is deducted as the examination location.

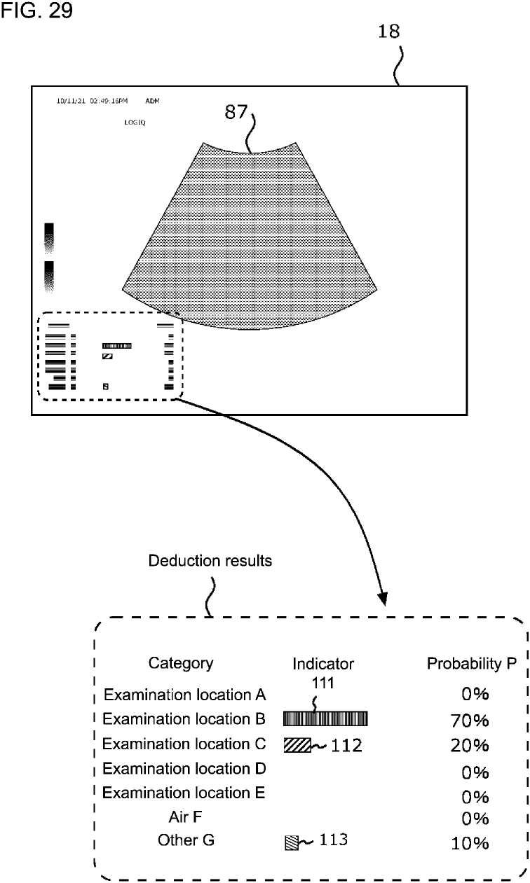

FIG. 29 is a diagram illustrating another example of the deduction results displayed on the display monitor 18.

In FIG. 29, the probability P for the examination location B is 70%. Therefore, the probability of the examination location B is within the range TH1 ≦ P ≦ TH2, so an indicator 111 for the examination location B is shown in yellow. In addition, the probability P of the examination location C is 20% and the probability P of other G is 10%. Therefore, the probabilities P of the examination location C and other G are within the range P < TH1, so an indicator 112 for the examination location C and an indicator 113 for other G are shown in red. Moreover, a length of the indicators 111, 112, and 113 are displayed depending on a value of the probability P. Thus, the user can visually recognize whether a probability of an examination location being included in a category is high.

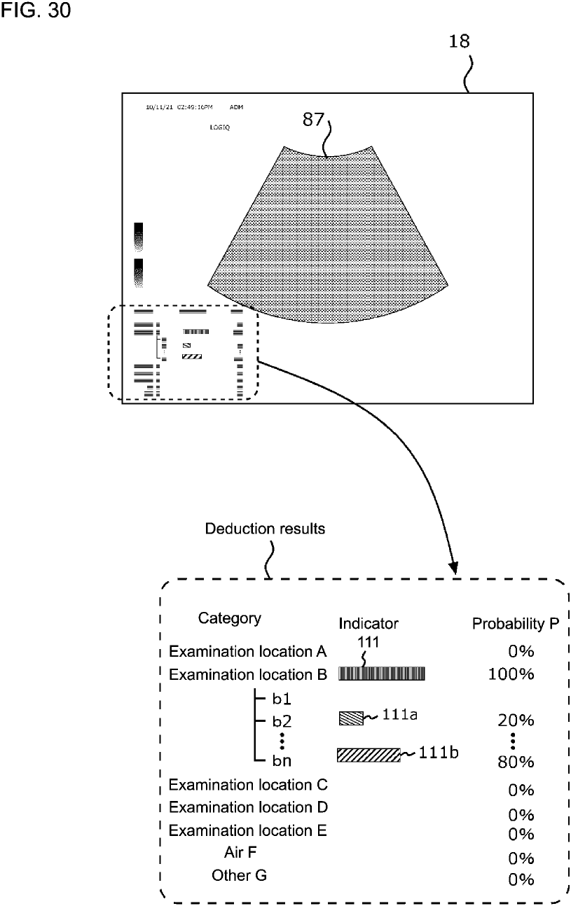

FIG. 30 is an example of deduction results of an examination location displayed in further detail.

The examination location B includes an n number of sub-examination locations b1 to bn. Furthermore, a probability of a location shown by the input image 83 being classified into each sub-examination location is displayed in the deduction results. Therefore, in the display example in FIG. 30, by checking a display screen, the user 51 is able to recognize which sub-examination location among the n number of sub-examination locations b1 to bn included in the examination location B has a highest probability of being the location of the ultrasonic image 87.

Note that the ultrasonic image 87 displayed in FIGS. 28 to 30 is not limited to a B-mode image, and an ultrasonic image of another mode may be displayed. An example of the ultrasonic diagnostic device in which a color image is displayed showing blood flow in color is described below.

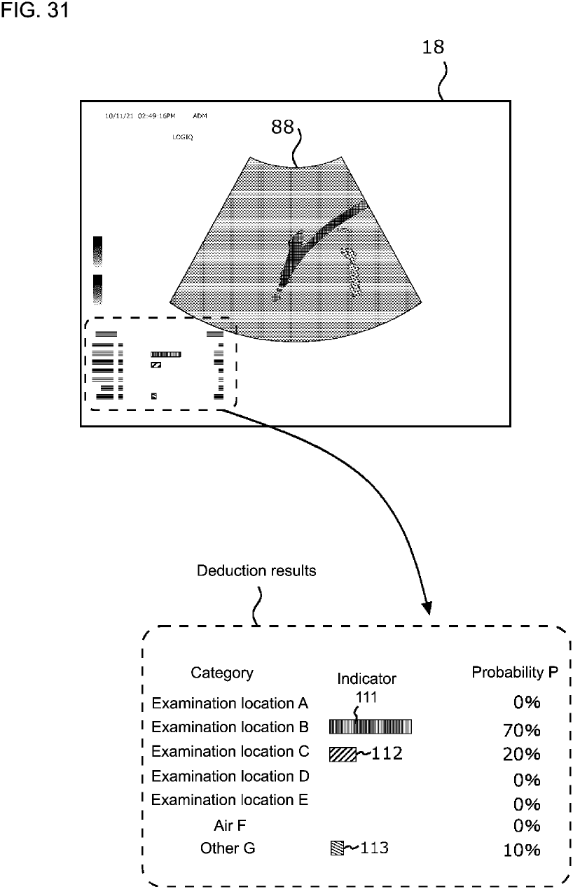

FIG. 31 is an example in which a color image 88 is displayed.

When the color image 88 is displayed, the user 51 operates the user interface 10 to activate a color mode for displaying the color image 88. When the color mode is activated, the processor 7 displays a color image 88 in which blood flow shown in color is weighted in the ultrasonic image acquired before or after the color mode is activated. Therefore, the user 51 can check blood flow dynamics via the color image 88.

Moreover, when the color mode is activated, the processor 7 can display deduction results on the display screen. Thus, the user 51 can check deduction results for any of a plurality of ultrasonic images.

Note that ultrasonic image and deduction results can also be displayed on the touch panel 28.

### Embodiment 9

In Embodiment 9, an example is described where a user sets, prior to examination of a subject or during the examination, the ultrasonic diagnostic device to activate via a preset recommendation mode that recommends preset changes to the user 51 or via a separate mode in which this preset recommendation mode is not executed.

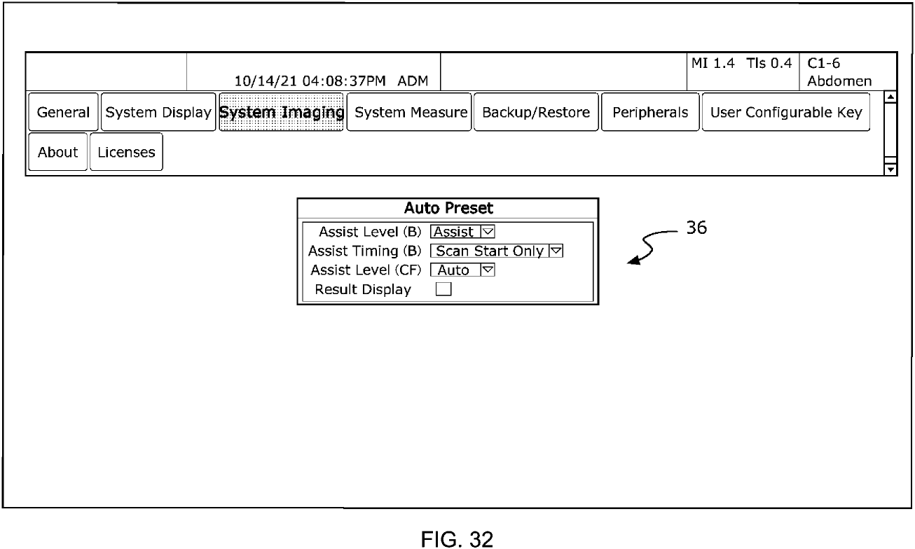

FIG. 32 is a diagram illustrating an example of a settings screen for setting an operating mode of the ultrasonic diagnostic device.

By operating the user interface, the user can display a settings window 36 for setting an operating mode of the ultrasonic diagnostic device 1 on the display monitor 18 or the touch panel 28. In the settings window 36, “Assist Level (B)”, “Assist Timing (B)”, “Assist Level (CF)”, and “Result Display” are displayed.

“Assist Level (B)” indicates an assistance level executed for the user 51 when a B-mode image is acquired. The “Assist Level (B)” includes three assistance levels (Auto/Assist/OFF). Auto indicates an automatic mode where the ultrasonic diagnostic device automatically changes a preset without using the preset recommendation mode. Assist indicates use of the preset recommendation mode. OFF indicates that the automatic mode and the preset recommendation mode are turned off.

“Assist Timing (B)” indicates timing for executing assistance when a B-mode image is acquired. “Assist Timing (B)” includes three assistance levels (All the time / Scan start / Exam start). “All the time” indicates execution of assistance set by “Assist Level (B)” from a start to an end of the examination of the subject. “Scan start” indicates execution of assistance set by “Assist Level (B)” from a start to an end of a scan of the subject. “Exam start” indicates execution of assistance set by “Assist Level (B)” until the scan of the subject 53 is started.

“Assist Level (CF)” indicates an assistance level executed for the user 51 when a color velocity image is acquired. Similarly to “Assist Level (B)”, “Assist Level (CF)” includes three assistance levels (Auto/Assist/OFF).

“Result Display” is an indicator as to whether deduction results (see FIGS. 28 to 31) are displayed. When “Result Display” is activated, the deduction results can be displayed.

Therefore, the user can set an assistance level according to their preference.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. An ultrasonic image display system, comprising: an ultrasonic probe, a user interface, a display, and one or more processors for communicating with the ultrasonic probe, the user interface, and the display, wherein the one or more processors execute operations including: selecting a preset used in examination from among a plurality of presets set for a plurality of examination locations based on a signal input through the user interface; deducing an examination location of a subject using a trained model by inputting to the trained model an input image created based on an ultrasonic image obtained by scanning the subject via the ultrasonic probe; determining whether to recommend that a user change the selected preset to a preset of the deduced examination location based on the selected preset examination location and the deduced examination location; and displaying a message on the display recommending that the user change the preset when it is determined that a preset change should be recommended to the user.

2. An ultrasonic image display system, wherein if the deduced examination location does not match the selected preset examination location, the one or more processors make a determination to recommend a preset change to the user, and if the deduced examination location does match the selected preset examination location, the one or more processors make a determination not to recommend a preset change to the user.

3. The ultrasonic image display system according to claim 2, wherein deducing an examination location of the subject includes deducing into which category among a plurality of categories, including a plurality of examination locations, a location of the input image is to be classified, obtaining a probability of the location shown by the input image being classified into each category, and deducing the examination location of the subject based on the probability.

4. The ultrasonic image display system according to claim 3, wherein the one or more processors execute an operation including determining to recommend a preset change to the user when the probability obtained for the deduced examination location is within a first threshold and a second threshold greater than the first threshold, and when the deduced examination location does not match the selected preset examination location.

5. The ultrasonic image display system according to claim 4, wherein when the probability is lower than the first threshold, the one or more processors do not recommend a preset change.

6. The ultrasonic image display system according to claim 4, wherein when the probability is greater than the second threshold and the deduced examination location does not match the selected preset examination location, the one or more processors change the selected preset to a preset for the deduced examination location.

7. The ultrasonic image display system according to claim 1, wherein when transmission and reception of ultrasonic waves is interrupted by a prescribed operation of the user, the one or more processors execute an operation including changing the selected preset to a preset for the deduced examination location.

8. The ultrasonic image display system according to claim 7, wherein the prescribed operation is a freeze operation, a screen storing operation, or a depth change operation.

9. The ultrasonic image display system according to claim 1, wherein when a preset is changed by the user, the one or more processors stops deducing the examination location.

10. The ultrasonic image display system according to claim 9, wherein after a location subject is started, the one or more processors execute an operation including determining whether the preset was changed by the user.

11. The ultrasonic image display system according to claim 9 wherein after the preset change is recommended to the user, the one or more processors execute an operation including determining whether the preset was changed by the user.

12. The ultrasonic image display system according to claim 7, wherein in response to a prescribed operation by the user, the one or more processors change the selected preset to the preset for the deduced examination location.

13. The ultrasonic image display system according to claim 6, wherein the one or more processors execute an operation of weighting the probability.

14. The ultrasonic image display system according to claim 3, wherein the one more processors operate such that the deduction results including the plurality of categories and a probability of a location indicated by the input image being classified into each category are displayed.

15. The ultrasonic image display system according to claim 14, wherein the deductions results include an indicator corresponding to a value of the probability.

16. The ultrasonic image display system according to claim 15, wherein the indicator is displayed in a color depending on the value of the probability or a length is displayed depending on the value of the probability.

17. The ultrasonic image display system according to claim 14, wherein a first examination location included in the plurality of categories includes a plurality of sub-examination locations, the deduction results include the plurality of sub-examination locations, and the location shown by the input image is classified into each sub-examination location.

18. The ultrasonic image display system according to claim 14, wherein the one or more processors display the ultrasonic image on the display.

19. The ultrasonic image display system according to claim 14, wherein an operating mode of the ultrasonic image display system includes a color mode for displaying a color image in which blood flow is shown in color, and when the color mode is activated, the one or more processors display the color image and the deduction results on the display.

20. Non-transitory storage media that can be read non-temporally by one or a plurality of computers on which one or a plurality of commands are stored that can be executed by one or more processors that communicate with an ultrasonic probe, a user interface, and a display, wherein the one or a plurality of commands execute operations including: selecting a preset used in examination from among a plurality of presets set for a plurality of examination locations based on a signal input through the user interface; deducing an examination location of a subject using a trained model by inputting, to the trained model, an input image created based on an ultrasonic image obtained by scanning the subject via the ultrasonic probe; determining whether to recommend that a user change the selected preset to a preset of the deduced examination location based on the selected preset examination location and the deduced examination location; and displaying a message on the display recommending that the user change the preset when it is determined that a preset change should be recommended to the user.
